(19) United States
(12) Patent Application Publication
(10) Pub. No.: US 2016/0077107 A1
(43) Pub. Date: Mar. 17, 2016

(54) PERSONALISED MEDICINE

(71) Applicant: STICHTING KATHOLIEKE UNIVERSITEIT, Nijmegen (NL)

(72) Inventors: Leonardus Antonius Bernardus Joosten, Beuningen (NL); Mihai Gheorghe Netea, Nijmegen (NL); Johannes Willem Maarten van der Meer, Nijmegen (NL)

(21) Appl. No.: 14/787,928

(22) PCT Filed: May 2, 2014
(86) PCT No.: PCT/NL2014/050282

§ 371 Date: Oct. 29, 2015

(30) Foreign Application Priority Data
May 2, 2013 (EP) 13166244.7

(51) Int. Cl. G01N 33/68; C07K 14/705; C07K 16/24; A61K 38/05; G01N 33/564

(52) U.S. Cl. CPC G01N 33/6869; A61K 38/05; G01N 33/6866; G01N 33/6893; G01N 33/6863; G01N 33/564; G01N 33/6896; C07K 16/241; C07K 14/70578; C07K 2317/31; G01N 2800/065; G01N 2800/52; G01N 2800/102; G01N 2800/285; G01N 2800/107; C07K 2319/30; C07K 2317/76; A61K 38/00

(57) ABSTRACT

The present invention relates to a method for assessing the efficacy of an inhibitor of a pro-inflammatory cytokine and/or of B cells in a subject and a method for treating said subject with said inhibitor provided the efficacy of said inhibitor has been determined as sufficient.

### FIELD OF THE INVENTION

The present invention relates to the field of diagnostics and therapeutics. It provides a method for assessing the efficacy of an inhibitor of a pro-inflammatory cytokine and/or an inhibitor of B cells and thereby selecting the best possible treatment for a subject suspected of having an autoimmune and/or inflammatory disease and/or condition.

### BACKGROUND OF THE INVENTION

Crohn's disease and ulcerative colitis are inflammatory bowel diseases (IBD) in which the dysregulated inflammatory reaction in the mucosa of the intestinal tract plays the major role in pathogenesis. Under physiologic conditions there is a balance between pro-inflammatory cytokines (e.g. TNFα or IL-1β) and anti-inflammatory cytokines (e.g. IL-10 or TGFβ). Interleukin-1β is one of the most important pro-inflammatory mediators of the innate immune system, and its role in the pathogenesis of IBD has been proved extensively (1).

Autophagy is one of the main processes that regulates secretion of IL-1β (2), and autophagy gene polymorphisms (e.g. ATG16L1, IRGM) have been shown to be associated with Crohn's disease (3). Recently, we have demonstrated that the T300A ATG16L1 polymorphism strongly modulates IL-1β production, and the risk variant is associated with an increased synthesis and release of the cytokine (4). The production of IL-1β is highly regulated by intracellular protein platforms called the inflammasomes (5,6). The inflammasomes that are linked to IL-1β production contain pro-caspase-1, the critical enzyme, when activated for intracellular cleavage of pro-IL-1β (7). We have assessed whether a specific caspase-1 inhibitor can block the processing and release of IL-1β, and can thus have beneficial effects on the inflammatory process in IBD. In addition, we have explored whether caspase-1 inhibitors are more effective in individuals with high production capacity of IL-1β on the one hand, and in those bearing the T300A ATG16L1 polymorphism on the other hand.

Surprisingly, we noted that the intrinsic capacity to produce cytokine was different in the screened subjects, and the effect of the anti-inflammatory agent depended on the cytokine production. It reveals that we could distinguish between high, intermediate and low producers of said cytokine. A high producer for said cytokine may be a high, intermediate or low producer for another cytokine. These results leads to the idea that it is possible to identify individual profiles of cytokine production and thereby stratify individual patient with (auto)inflammatory diseases poor responder to a certain anti-inflammatory biological therapy and a good responder to another biological therapy, based on stratification of the patients in high and low cytokine producers. This stratification is expected to be applicable for all treatments based on biological therapies that are currently used. The stratification of patients may be of great importance for both the patients that would be spared treatments that are unlikely to be successful and that have potential important side-effects, and for the healthcare system due to the substantial cost-saving aspect of such a personalized approach.

### DESCRIPTION OF THE INVENTION

### Assessing Method

### First Method: Assessing the Efficacy of an Inhibitor of a Pro-Inflammatory Cytokine

In a first aspect, the invention relates to a method for assessing the efficacy of an inhibitor of a pro-inflammatory cytokine in a subject, said method comprising the steps of:
(a) obtaining a sample from said subject,
(b1) contacting said sample with a compound able to induce the production of a pro-inflammatory cytokine in said sample and
(b2) contacting said sample with said inhibitor of said pro-inflammatory cytokine in said sample,
(c) determining the profile or expression level of said pro-inflammatory cytokine in said sample at the end of step (b1) and (b2) and
(d) assessing the efficacy of said inhibitor as sufficient when at the end of step (b1) a detectable profile or expression level or an increase of the profile or expression level of said pro-inflammatory cytokine has been detected and when at the end of step (b2) a detectable decrease of profile or the expression level of said pro-inflammatory cytokine has been detected.

A pro-inflammatory cytokine is a cytokine that is able to promote systemic inflammation. A pro-inflammatory cytokine is preferably involved in or linked with or a consequence of an inflammatory and/or autoimmune disease or condition as defined herein. A pro-inflammatory cytokine is preferably selected from the group consisting of IL-1β, TNFα or IFNγ, IL-6, IL-12, IL-17, IL-23, IL-5. IL-1β is a preferred pro-inflammatory cytokine. IL-1β is known to be involved in Crohn's disease.

Such an inhibitor of a pro-inflammatory cytokine may be a compound able to inhibit the production of said pro-inflammatory cytokine and/or able to decrease the expression level of said pro-inflammatory cytokine and/or able to decrease an activity of said pro-inflammatory cytokine and/or able to inhibit a receptor of said cytokine and/or able to compete for the binding of said cytokine to its receptor. Such inhibitor may inhibit a chain of said cytokine receptor and/or may compete for the binding of said cytokine to its receptor by targeting one chain of its receptor. Such inhibitor may exhibit this inhibition and/or decrease in a subject or in a sample of said subject as defined later herein. An inhibitor of IL-6 may be an inhibitor of IL-6R. An inhibitor of IL-6R is preferably an inhibitor of one chain of the IL-6R, more preferably an inhibitor of the alpha chain of the IL-6R. An inhibitor of IL-17 may be an inhibitor of IL-17A or of IL-17F. An inhibitor of IL-12 may be an inhibitor of IL-12P40. Preferred inhibitors of pro-inflammatory cytokines are identified in table 2.

Several inhibitors of a pro-inflammatory cytokine are already known and have been developed and some are still being developed for treating, delaying, curing, preventing an inflammatory and/or autoimmune disease or condition. Examples of preferred inflammatory and/or autoimmune disease or condition include Inflammatory Bowel Disease (IBD), Rheumatoid arthritis (RA), other RA-like diseases, Crohn disease, Multiple sclerosis (MS), Psoriasis, Hidradenitis Suppurativa, Chronic Obstructive Pulmonary Disease (COPD), Sarcoidosis, Gout, Wegener Disease, type 2 diabetes, atherosclerosis, Lyme disease, Sepsis, Asthma, Ulcerative colitis, ankylosing spondylitis. Other RA-like diseases include Sporiatic arthritis, ankylosing spondylitis or juvenile arthritis. Preferred inflammatory and/or autoimmune diseases or conditions are identified in table 1.

Examples of inhibitors of a pro-inflammatory cytokine include inhibitory antibodies raised against such pro-inflammatory cytokine as Ilaris (i.e. canakinumab) from Novartis which is a human monoclonal antibody raised against IL-1β, Humira (i.e. Adalimumab) an inhibitor of TNFα from Abbott or Enbrel (i.e. Etanercept) another inhibitor of TNFα from Wyeth/Pfizer, or Tocilizumab an inhibitor of IL-6R from Roche. Small molecules (or peptidomimetics) inhibiting such pro-inflammatory cytokine as the caspase 1 inhibitor VRT (Vertex) In a later section, more information is provided as to how generate an inhibitory antibody or a peptidomimetic. The caspase 1 inhibitor VRT is known to inhibit the production of IL-1β and said caspase 1 inhibitor has already been used in the treatment of the Crohn disease as explained in the experimental part. Tocilizumab is already used in the treatment of Crohn and RA. Enbrel is already used in the treatment of RA, ankylosing spondylitis, and psoriatic arthritis.

### Second Method: Assessing the Efficacy of an Inhibitor of B Cells

In another aspect, the invention relates to a method for assessing the efficacy of an inhibitor of B cells in a subject, said method comprising the steps of:
(a) obtaining a sample from said subject,
(b1) contacting said sample with a compound able to induce the production of B cells in said sample and
(b2) contacting said sample with said inhibitor of said B cells in said sample,
(c) determining the number of B cells in said sample at the end of step (b1) and (b2) and
(d) assessing the efficacy of said inhibitor as sufficient when at the end of step (b1) a detectable number or an increase of the number of said B cells has been detected and when at the end of step (b2) a detectable decrease of the number of said B cells has been detected.

B cells as pro-inflammatory cytokine may able to promote systemic inflammation. B cells may be involved in or linked with or be a consequence of an inflammatory and/or autoimmune disease or condition as defined herein. B cells may be identified in a sample by assessing the presence of a B cell specific marker by techniques known to the skilled person. A preferred technique if the marker is expressed at the cell surface is FACS (Fluorescence-activated cell sorting) analysis using a specific antibody recognizing (part of) the extracellular domain of said marker. A preferred B cell specific marker is CD20 or CD19. A preferred commercial available antibody against CD20 to be used in such FACS analysis is Anti-CD20, clone 2H7 from Millipore.

It is clear to the skilled person that a B cell marker could be used in the invention in order to assess the number of B cells present in a sample. It could also be a target of the inhibitor of B cells as identified herein.

Such an inhibitor of B cells may be a compound able to inhibit the production of said B cells and/or able to decrease the number of said B cells and/or able to decrease an activity of said B cells. An inhibitor of B cells may also be called a compound able to deplete/capture/inactivate B cells and/or deplete/capture/inactivate antibodies produced by such B cells. An activity of a B cell may be the production of a pro-inflammatory cytokine as IL-6 or IL-10 or may be to promote the production of a pro-inflammatory cytokine by other cells, such as T helper cells (Th17). Th17 are known to be able to produce IL-17 as a pro-inflammatory cytokine.

Several inhibitors of B cells are already known and have been developed and some are still being developed for treating, delaying, curing, preventing an inflammatory and/or autoimmune disease or condition. A preferred inhibitor of B cell is an inhibitor of CD20, more preferably as identified in table 2: Rituximab (Roche, CH), ofatumumab (GSK, UK), veltuzumab (Takeda, JP) or ocrelizumab (Roche CH). Another preferred inhibitor of B cell is an inhibitor of CD19. A preferred inhibitor or CD19 is GBR 401 (Glenmark Pharmaceuticals, CH).

Examples of preferred inflammatory and/or autoimmune disease or condition have already been defined herein. More preferred inflammatory and/or autoimmune disease or condition for which B cells may be involved in or linked with or be a consequence of such disease or condition include Rheumatoid arthritis (RA) and Multiple sclerosis (MS). Preferred inflammatory and/or autoimmune diseases or conditions are identified in table 1.

In a preferred embodiment, the first method (i.e. one assessing the efficacy of an inhibitor of a pro-inflammatory cytokine) and the second method (i.e. one assessing the efficacy of an inhibitor of B cells) are applied on the same subject. Below each feature of the first method is further defined. Unless otherwise indicated, each feature of the first method could be applied on the second method.

Therefore, the invention relates to a method for assessing the efficacy of an inhibitor of a pro-inflammatory cytokine and/or for assessing the efficacy of an inhibitor of B cells in a subject, said method comprising the steps of:
(a) obtaining a sample from said subject,
(b1) contacting said sample with a compound able to induce the production of a pro-inflammatory cytokine in said sample and/or contacting said sample with a compound able to induce the production of B cells in said sample and
(b2) contacting said sample with said inhibitor of said pro-inflammatory cytokine in said sample and/or contacting said sample with said inhibitor of said B cells in said sample,
(c) determining the profile or expression level of said pro-inflammatory cytokine and/or determining the number of B cells in said sample at the end of step (b1) and (b2) and
Step (d):
(d1) assessing the efficacy of said inhibitor of a pro-inflammatory cytokine as sufficient when at the end of step (b1) a detectable profile or expression level or an increase of the profile or expression level of said pro-inflammatory cytokine has been detected and when at the end of step (b2) a detectable decrease of profile or the expression level of said pro-inflammatory cytokine has been detected and/or
(d2) assessing the efficacy of said inhibitor of B cells as sufficient when at the end of step (b1) a detectable number or an increase of the number of said B cells has been detected and when at the end of step (b2) a detectable decrease of the number of said B cells has been detected.

The method of the invention (i.e. first and/or second method) could be carried out before a subject is being treated for a given autoimmune and/or inflammatory disease and/or condition. It is also encompassed by the present invention to carry out the method of the invention once such treatment has started. In such a case, the efficacy of the current treatment (i.e. inhibitor of a pro-inflammatory cytokine in a first method and inhibitor of B cells in a second method) may be compared with the one of other possible treatment (i.e. other inhibitor of a pro-inflammatory cytokine in a first method and inhibitor of B cells in a second method). If the method of the invention indicates that the efficacy of other treatments is expected to be better than the one of the current treatment, the type of treatment administered to said subject may be modified and the inhibitor with the best efficacy may be chosen.

In the context of the invention, a subject may be a human being or an animal. The method (i.e. first and/or second method) may be applied as often as necessary in a subject. If the subject is a human being, the subject may be a person suspected to have a high risk of having or developing an autoimmune and/or inflammatory disease or condition, for example due its genetic background.

Therefore in a preferred embodiment, the invention provides a method for assessing the efficacy of an inhibitor of a pro-inflammatory cytokine in a subject suspected to suffer from an autoimmune and/or inflammatory disease or condition, wherein said pro-inflammatory cytokine is selected from the group consisting of: IL-1-β, IL-6, IL-17, IL-23, IL-12, IL-5, TNFα and IFNγ and said method comprising the steps of:
(a) obtaining a sample from said subject,
(b1) contacting said sample with a compound able to induce the production of a pro-inflammatory cytokine in said sample and
(b2) contacting said sample with said inhibitor of said pro-inflammatory cytokine in said sample,
(c) determining the profile or expression level of said pro-inflammatory cytokine in said sample at the end of step (b1) and (b2) and
(d) assessing the efficacy of said inhibitor of said pro-inflammatory cytokine as sufficient when at the end of step (b1) a detectable expression level or an increase of the expression level of said pro-inflammatory cytokine has been detected and when at the end of step (b2) a detectable decrease of the expression level of said pro-inflammatory cytokine has been detected.

Table 1 provides an overview of several autoimmune and/or inflammatory diseases and/or conditions and main pro-inflammatory cytokines known to be involved in said diseases and/or conditions. Table 1 also provides an overview of several autoimmune and/or inflammatory diseases and/or conditions wherein B cells are suspected to be involved or to play a role. Table 2 gives an overview of some of the known inhibitors of some pro-inflammatory cytokines. Table 2 also provides an overview of some known inhibitors of B cells.

In an embodiment, a method of the invention as earlier defined herein (i.e. for assessing the efficacy of an inhibitor of a pro-inflammatory cytokine and/or for assessing the efficacy of a B cell inhibitor in a subject preferably, wherein said subject is suspected to suffer from an autoimmune and/or inflammatory disease or condition) is such that:
the autoimmune and/or inflammatory disease or condition is: RA (rheumatoid arthritis) and/or
the pro-inflammatory cytokine is selected from the group consisting of: TNFα, IL-1β, IL-6, IL-12, IL-17 and IL-23 and/or
the B cell marker targeted by the B cell inhibitor is CD20 or CD19.

Preferably, if the pro-inflammatory cytokine is IL-12, the inhibitor of IL-12 is an inhibitor of IL-12p40. Such preferred inhibitor is Ustekinumab (Janssen-Cilag, BE).

Preferably, if the pro-inflammatory cytokine is IL-17, the inhibitor of IL-17 is an inhibitor of IL-17A. Preferred inhibitor of IL-17A, is Brodalumab (Amgen, USA); Ixekizumab (Lilly (Eli), USA) or Secukinumab (Novartis, CH).

Preferably, if the pro-inflammatory cytokine is IL-23, the inhibitor of IL-23 is Ustekinumab (Janssen-Cilag, BE).

Preferably, if the pro-inflammatory cytokine is CD20, the inhibitor of CD20 is Rituximab (Roche, CH).

Preferably, if the pro-inflammatory cytokine is IL-6, the inhibitor of IL-6 is Tocilizumab (Roche, CH).

Preferably, if the pro-inflammatory cytokine is IL-1β, the inhibitor of IL-1β is Anakinra (IL-1Rα) (Sobi, SE); Ilaris (Anti-IL-1b) (Novartis, CH).

Preferably, if the pro-inflammatory cytokine is TNFα, the inhibitor of TNFα is Embrel (Amgen and Whyett, USA); Humira (Abbott, USA); Infliximab (Centocor Pharmaceuticals (Johnson & Johnson), USA); Golimumab (MSD).

A preferred CD20 inhibitor is Rituximab (Roche, CH), ofatumumab (GSK, UK), veltuzumab (Takeda, JP) or ocrelizumab (Roche CH). Another preferred inhibitor of B cell is an inhibitor of CD19. A preferred inhibitor or CD19 is GBR 401 (Glenmark Pharmaceuticals, CH).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is: another RA-like disease and/or
the pro-inflammatory cytokine is TNFα.

Preferably, if the pro-inflammatory cytokine is TNFα, the inhibitor of TNFα is Embrel (Amgen and Whyett, USA); Humira (Abbott, USA); Infliximab (Centocor Pharmaceuticals (Johnson&Johnson), USA); Golimumab (MSD).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is ulcerative colitis and/or
the pro-inflammatory cytokine is TNFα.

Preferably, if the pro-inflammatory cytokine is TNFα, the inhibitor of TNFα is Embrel (Amgen and Whyett, USA); Humira (Abbott, USA); Infliximab (Centocor Pharmaceuticals (Johnson&Johnson), USA); Golimumab (MSD).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is Crohn disease and/or
the pro-inflammatory cytokine is selected from the group consisting of: TNFα, IL-1β, IL-12, IL-17 and IL-23.

Preferably, if the pro-inflammatory cytokine is IL-12, a preferred inhibitor of IL-12 is an inhibitor of IL-12p40. A preferred inhibitor of IL-12p40 is Ustekinumab (Janssen-Cilag, BE).

Preferably, if the pro-inflammatory cytokine is IL-17, a preferred inhibitor of IL-17 is an inhibitor of IL-17A. A preferred inhibitor of IL-17A is Brodalumab (Amgen, USA); Ixekizumab (Lilly (Eli), USA) or Secukinumab (Novartis, CH).

Preferably, if the pro-inflammatory cytokine is IL-23, the inhibitor of IL-23 is Ustekinumab (Janssen-Cilag, BE).

Preferably, if the pro-inflammatory cytokine is IL-1β, the inhibitor of IL-1β is Anakinra (IL-1Rα) (Sobi, SE); Ilaris (Anti-IL-1b) (Novartis, CH).

Preferably, if the pro-inflammatory cytokine is TNFα, the inhibitor of TNFα is Embrel (Amgen and Whyett, USA); Humira (Abbott, USA); Infliximab (Centocor Pharmaceuticals (Johnson&Johnson), USA); Golimumab (MSD).

In an embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is: psoriasis and/or
the pro-inflammatory cytokine is selected from the group consisting of: TNFα, IL-12, IL-17 and IL-23.

Preferably, if the pro-inflammatory cytokine is IL-12, a preferred inhibitor of IL-12 is an inhibitor of IL-12p40. A preferred inhibitor of IL-12p40 is Ustekinumab (Janssen-Cilag, BE).

Preferably, if the pro-inflammatory cytokine is IL-17, a preferred inhibitor of IL-17 is an inhibitor of IL-17A. A preferred inhibitor of IL-17A is Brodalumab (Amgen, USA); Ixekizumab (Lilly (Eli), USA) or Secukinumab (Novartis, CH).

Preferably, if the pro-inflammatory cytokine is IL-23, the inhibitor of IL-23 is Ustekinumab (Janssen-Cilag, BE).

Preferably, if the pro-inflammatory cytokine is TNFα, the inhibitor of TNFα is Embrel (Amgen and Whyett, USA); Humira (Abbott, USA); Infliximab (Centocor Pharmaceuticals (Johnson&Johnson), USA); Golimumab (MSD).

In another embodiment, a method of the invention as earlier defined herein (i.e. for assessing the efficacy of an inhibitor of a pro-inflammatory cytokine and/or for assessing the efficacy of a B cell inhibitor in a subject preferably, wherein said subject is suspected to suffer from an autoimmune and/or inflammatory disease or condition) is such that:
the autoimmune and/or inflammatory disease or condition is: MS (multiple Sclerosis) and/or
the pro-inflammatory cytokine is selected from the group consisting of: IL-1β, and IL-17 and/or
the B cell marker targeted by the B cell inhibitor is CD20 or CD19.

Preferably, if the pro-inflammatory cytokine is IL-17, a preferred inhibitor of IL-17 is an inhibitor of IL-17A or an inhibitor of IL-17F. A preferred inhibitor of IL-17A is Brodalumab (Amgen, USA); Ixekizumab (Lilly (Eli), USA) or Secukinumab (Novartis, CH).

Preferably, if the pro-inflammatory cytokine is CD20, the inhibitor of CD20 is Rituximab (Roche, CH).

Preferably, if the pro-inflammatory cytokine is IL-1β, the inhibitor of IL-1β is Anakinra (IL-1Rα) (Sobi, SE); Ilaris (Anti-IL-1b) (Novartis, CH).

A preferred CD20 inhibitor is Rituximab (Roche, CH), ofatumumab (GSK, UK), veltuzumab (Takeda, JP) or ocrelizumab (Roche CH). Another preferred inhibitor of B cell is an inhibitor of CD19. A preferred inhibitor or CD19 is GBR 401 (Glenmark Pharmaceuticals, CH).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is: asthma and/or
the pro-inflammatory cytokine is selected from the group consisting of: IL-5 and IFNγ.

Preferably, if the pro-inflammatory cytokine is IL-5, the inhibitor of IL-5 is Mepolizumab (GSK, UK).

Preferably, if the pro-inflammatory cytokine is IFNγ, the inhibitor of IFNγ is Immukine (Boehringer Ingelheim, DE).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is: sepsis and/or
the pro-inflammatory cytokine is selected from the group consisting of: IL-1β and IFNγ.

Preferably, if the pro-inflammatory cytokine is IL-1β, the inhibitor of IL-1β is Anakinra (IL-1Rα) (Sobi, SE); Ilaris (Anti-IL-1β) (Novartis, CH).

Preferably, if the pro-inflammatory cytokine is IFNγ, the inhibitor of IFNγ is Immukine (Boehringer Ingelheim, DE).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is: gout and/or
the pro-inflammatory cytokine is IL-1β.

Preferably, if the pro-inflammatory cytokine is IL-1β, the inhibitor of IL-1β is Anakinra (IL-1Rα) (Sobi, SE); Ilaris (Anti-IL-1β) (Novartis, CH).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is: the Lyme disease and/or
the pro-inflammatory cytokine is selected from the group consisting of: IL-1β and IL-17.

Preferably, if the pro-inflammatory cytokine is IL-17, a preferred inhibitor of IL-17 is an inhibitor of IL-17A. A preferred inhibitor of IL-17A is Brodalumab (Amgen, USA); Ixekizumab (Lilly (Eli), USA) or Secukinumab (Novartis, CH).

Preferably, if the pro-inflammatory cytokine is IL-1β, the inhibitor of IL-1β is Anakinra (IL-1Rα) (Sobi, SE); Ilaris (Anti-IL-1b) (Novartis, CH).

In another embodiment, a method of the invention as earlier defined herein (i.e. for assessing the efficacy of an inhibitor of a pro-inflammatory cytokine in a subject preferably, wherein said subject is suspected to suffer from an autoimmune and/or inflammatory disease or condition) is such that:
the autoimmune and/or inflammatory disease or condition is Type II Diabetes and/or
the pro-inflammatory cytokine is selected from the group consisting of: TNFα and IL-1β.

Preferably, if the pro-inflammatory cytokine is IL-1β, the inhibitor of IL-1β is Anakinra (IL-1Rα) (Sobi, SE); Ilaris (Anti-IL-1b) (Novartis, CH).

Preferably, if the pro-inflammatory cytokine is TNFα, the inhibitor of TNFα is Embrel (Amgen and Whyett, USA); Humira (Abbott, USA); Infliximab (Centocor Pharmaceuticals (Johnson&Johnson), USA); Golimumab (MSD).

Accordingly a preferred method is applied to the following autoimmune and/or inflammatory disease or condition and/or to the following pro-inflammatory cytokine and/or to the following B cell marker:

i. RA and/or the pro-inflammatory cytokine is selected from the group consisting of: TNFα, IL-1β, IL-6, IL-12, IL-17 and IL-23 and/or the B cell marker is CD20 and/or CD19,
ii. another RA-like disease and/or the pro-inflammatory cytokine is TNFα,
iii. ulcerative colitis and/or the pro-inflammatory cytokine is TNFα,
iv. Crohn disease and/or the pro-inflammatory cytokine is selected from the group consisting of: TNFα, IL-1β, IL-12, IL-17 and IL-23,
v. psoriasis and/or the pro-inflammatory cytokine is selected from the group consisting of: TNFα, IL-12, IL-17 and IL-23,
vi. MS and/or the pro-inflammatory cytokine is selected from the group consisting of: IL-1β and IL-17 and/or the B cell marker is CD20 and/or CD19,
vii. asthma and/or the pro-inflammatory cytokine is selected from the group consisting of: IL-5 and IFNγ,
viii. sepsis and/or the pro-inflammatory cytokine is selected from the group consisting of: IL-1β and IFNγ,
ix. gout and/or the pro-inflammatory cytokine is IL-1β,
x. Lyme disease and/or the pro-inflammatory cytokine is selected from the group consisting of: IL-1β and IL-17,
xi. Type II Diabetes and/or the pro-inflammatory cytokine is selected from the group consisting of: TNFα and IL-1β.

In step (a) of the first and second methods (i.e. assessing the efficacy of an inhibitor of a pro-inflammatory cytokine and assessing the efficacy of an inhibitor of B cells) a sample from said subject is obtained. A method of the invention is therefore an in-vitro or ex-vivo method and/or a non-invasive method. A sample preferably comprises or consists of a fluid obtained from a subject. More preferably, a fluid comprises or consists of or is derived from or is selected from: urine, blood, spinal cord fluid, saliva, semen, or bronchoalveolar lavage. A preferred fluid is, comprises, consists of or is derived from blood. Blood may be used as whole blood or diluted or purified before being further used. The dilution may be 1:4, 1:5 or 1:6 in culture medium or a buffered solution. A sample may comprise cells. A preferred sample comprises blood cells (i.e. B cells and/or T cells and/or B cells precursors and/or T cells precursors). Preferred cells include PBMC (Peripheral Blood Mononuclear Cells). A preferred sample comprises a fluid, more preferably comprises blood and even more preferably comprises PBMC. Depending on the identity of the sample, the sample may be cultured. For example if the sample comprises PBMC, it may be cultured in a suitable medium supplemented with suitable compounds known to the skilled person. Preferably, PBMC are cultured as explained in the experimental part.

A preferred sample of step a) or obtained in step a) of the first method (i.e. for assessing the efficacy of an inhibitor of a pro-inflammatory cytokine) does not comprise a pro-inflammatory cytokine or does not comprise detectable amounts thereof assessed as explained later herein (i.e. RT PCR or ELISA).

A preferred sample of step a) or obtained in step a) of the second method (i.e. for assessing the efficacy of an inhibitor of B cells) does not comprise B cells or does not comprise detectable numbers thereof assessed as explained herein (FACS analysis using a B cell marker). Specific B cells markers have already been identified herein (CD19 or CD20). A preferred commercial available antibody against CD20 to be used in such FACS analysis is Anti-CD20, clone 2H7 from Millipore.

It is encompassed by the present invention that each of the first and the second methods uses a distinct type of sample as identified herein. However, the same type of sample may be used in each method.

In step (b1) of the first method (i.e. assessing the efficacy of an inhibitor of a pro-inflammatory cytokine), said sample obtained in step (a) is contacted with a compound able to induce the production of a pro-inflammatory cytokine in said sample. In the context of the invention, a compound able to induce the production of a pro-inflammatory cytokine may be replaced by a compound able to increase the production of said cytokine. Any known compound able to induce the production of a pro-inflammatory cytokine may be used. For example, LPS, MDP, LPS/MDP, Pam3cys/MDP, poly I:C, flagellin or HK E. Coli are known to induce or increase the production of IL-1β (8).

In a preferred embodiment, the compound used to induce the production of a pro-inflammatory cytokine is specific for a given autoimmune and/or inflammatory disease or condition. Preferably, said compound is able to bind a receptor present on diseased cells or on cells of a diseased subject. It has been demonstrated that the use of such compound improves the sensitivity and/or specificity and/or predictability of the method of the invention compared to a method wherein a compound would be used that is not specific for the disease (see among other FIG. 5, example 7).

For Inflammatory Bowel Disease (IBD) or Crohn disease, such preferred compound is MDP (Muramyl dipeptide (Invivogen USA). Preferably 8 to 12 μg/ml of MDP is used. More preferably 10 μg/ml. MDP is a well-known NOD2 ligand and is regarded as disease specific (see example 3).

For MS, such preferred compound is Myelin Basic Protein or MOG peptides in combination with anti-CD3/CD28 (see example 7). A preferred anti-CD3 and a preferred anti-CD28 are from MACS miltenyi biotec (Germany). An anti-CD3 and an anti-CD28 could be each used in a concentration of at least 0.8, 0.9, 1, 1.1, 1.2 μg/ml. Preferred concentration for each of these antibodies is 1 μg/ml. MOG peptides have the following amino acid sequence: Met-Glu-Val-Gly-Trp-Tyr-Arg-Ser-Pro-Phe-Ser-Arg-Val-Val-His-Leu-Tyr-Arg-Asn-Gly-Lys. They could be purchased from Tocris Biosciences, Cat No. 2568).

For Gout, such preferred compound is Mono Sodium Urate (MSU) crystals and fatty acids (C16.0)) (see example 6) Preferably, MSU/C16.0 is used in concentration ranged from (280 μg/ml, 180 μM C16.0) (290 μg/ml, 190 μM C16.0) (300 μg/ml, 200 μM C16.0) (310 μg/ml, 210 μM C16.0). C16.0 could be purchased from Sigma Aldrich (USA). MSU could be prepared using techniques known to the skilled person.

For Lyme disease, such compound may be or may comprise a Borrelia antigen or a whole Borrelia cell or a part thereof or a lysate thereof.

For Sepsis, such compound may be or may comprise a Bacterial and/or a Fungal antigen or a whole Bacterial and/or fungal cell or a part thereof or a lysate thereof.

For Asthma, such compound may be Chitin and/or an Aspergillus antigen. For asthma such compound may comprise an Aspergillus antigen and/or a whole Aspergillus cell or a part thereof or a lysate thereof.

In step (b1) of the second method (i.e. assessing the efficacy of an inhibitor of B cells), said sample obtained in step (a) is contacted with a compound able to induce the production of B cells in said sample. In the context of the invention, a compound able to induce the production of B cells may be replaced by a compound able to increase the number of B cells and/or by a compound able to increase or activate an activity of such B cells. Any known compound able to induce or increase the production of B cells may be used. For example a compound known to induce or increase the production of B cells includes IL-5, IL-6 or IL-7.

In a preferred embodiment, the compound used to induce the production of B cells is specific for a given autoimmune and/or inflammatory disease or condition. Preferably, said compound is able to bind a receptor present on diseased cells or on cells of a diseased subject.

The (b1) contact step in the first and in the second methods (i.e. assessing the efficacy of an inhibitor of a pro-inflammatory cytokine and assessing the efficacy of an inhibitor of B cells) may have a duration of 1, 2, 3, 4, 5, 6, 7, 8, 12, 24, 30, 48, 60, 70, 80, 90, 93, 96, 100, 110 hours, or more. Preferably the contact has a duration of 4-96 hours, or 20-50 hours, or 24 hours, or 48 hours. This contact step may be a culture step in a culture medium such as RPMI 1640.

In step (b2) of the first and the second methods (i.e. assessing the efficacy of an inhibitor of a pro-inflammatory cytokine and assessing the efficacy of an inhibitor of B cells), said sample obtained in step (a) is contacted with an inhibitor of said pro-inflammatory cytokine (and/or with an inhibitor of B cells for the second method). The identity of said inhibitor of said pro-inflammatory cytokine has already been defined herein. The identity of said inhibitor of B cells has already been defined herein. As in step (b1), the contact may have a duration of 1, 2, 3, 4, 5, 6, 7, 8, 12, 24, 30, 48, 60, 70, 80, 90, 93, 96, 100, 110 hours, or more. Preferably the contact has a duration of 4-96 hours, or 20-50 hours, or 24 hours, or 48 hours. This contact step may be a culture step in a culture medium such as RPMI 1640. Usually the sample of step (a) is divided in at least two parts, three parts, four parts and on in each of these parts steps (b1) and (b2) are carried out. Steps (b1) and (b2) may be carried out sequentially or simultaneously, preferably sequentially. In case the first and the second methods are carried out sequentially or simultaneously, the sample of (a) may be divided in four parts: the first method being applied in two of these parts, the second method in remaining two parts.

It is also encompassed by the invention that if a distinct type of sample is used for the first versus second method, each sample may only be divided in two for carrying out steps (b1) and (b2) of each method.

In step (c) of the first method (i.e. assessing the efficacy of an inhibitor of a pro-inflammatory cytokine), the profile or expression level of said pro-inflammatory cytokine is determined in said sample at the end of step (b1) and (b2).

In the context of the invention, the expressions “profile”, “expression profile” or “profile of expression” may be replaced by “expression level” or “production level” or “activity level”. The profile of a pro-inflammatory cytokine may therefore refer to its production (encoding nucleic acid and/or protein levels) level and/or its activity level.

The assessment of the profile or the expression levels of said pro-inflammatory cytokine may be directly realised at the protein expression level (quantifying the amount of said proteins) and/or at the activity level (quantifying an activity of said protein) and/or indirectly by quantifying the amount of nucleotide sequences encoding said pro-inflammatory cytokine. The skilled person will understand that it is possible to isolate multiple isoforms of a pro-inflammatory cytokine depending on the subject or species to be tested.

In step (c) of the second method (i.e. assessing the efficacy of an inhibitor of B cells), the number of said B cells is determined in said sample at the end of step (b1) and (b2).

The number of B cells may also refer to an activity of said cell.

The number of B cells may be directly assessed at the cellular level (quantifying the amount of said cells) and/or at the activity level (quantifying an activity of said cells). The skilled person knows methods to assess number or activity of B cells. Number of B cells may be assessed using FACS technique as explained earlier herein. An activity of a B cell may be the production of pro-inflammatory cytokines (e.g. IL-6) or promotes the production of pro-inflammatory cytokines by other cells, e.g. T helper cells (Th17). The assessment of the production of a pro-inflammatory cytokine as IL-6 has been explained herein in the context of the first method. Therefore, in an embodiment of the second method, the profile or expression level of a pro-inflammatory cytokine is determined in said sample at the end of step (b1) and (b2). This profile or expression level is assessed the same way as described herein for the first method. The number of T helper cells 17 may be assessed as B cells using FACS analysis or by Cytokine flow phenotyping. Cytokine flow phenotyping allows to assess the intracellular expression of a marker said cells. Examples of markers of Th17 cells include IL-17A, IL-17F, IL-21, IL-22, CD4. Examples of compounds to be used for assessing the presence of these markers include:

For IL-17A: eBio64DEC17 FITC

### For IL-17F: SHLR17 PE

For IL-21: eBio3A3-N2 Alexa Fluor® 647

### For IL-22: 22URTI PerCP-eFluor® 710

For CD4: RPA-T4 eFluor® 450
All are from e-Biosciences, USA

A preferred nucleotide acid sequence encoding IL-1β comprises or consists of SEQ ID NO: 1. A preferred corresponding IL-1β amino acid sequence comprises or consist of SEQ ID NO: 2.

A preferred nucleotide acid sequence encoding IL-6 comprises or consists of SEQ ID NO:3. A preferred corresponding IL-6 amino acid sequence comprises or consists of SEQ ID NO:4.

A preferred nucleotide acid sequence encoding IL-17 comprises or consists of SEQ ID NO:5. A preferred corresponding IL-17 amino acid sequence comprises or consists of SEQ ID NO:6.

A preferred nucleotide acid sequence encoding IL-23 comprises or consists of SEQ ID NO:7. A preferred corresponding IL-23 amino acid sequence comprises or consists of SEQ ID NO:8.

A preferred nucleotide acid sequence encoding TNFα comprises or consists of SEQ ID NO:9. A preferred corresponding TNFα amino acid sequence comprises or consists of SEQ ID NO:10.

A preferred nucleotide acid sequence encoding IFNγ comprises or consists of SEQ ID NO:11. A preferred corresponding IFNγ amino acid sequence comprises or consists of SEQ ID NO:12.

A preferred nucleotide acid sequence encoding IL-12 comprises or consists of SEQ ID NO:25. A preferred corresponding IL-12 amino acid sequence comprises or consists of SEQ ID NO26.

A preferred nucleotide acid sequence encoding IL-5 comprises or consists of SEQ ID NO:29. A preferred corresponding IL-5 amino acid sequence comprises or consists of SEQ ID NO:30.

In a preferred embodiment, a pro-inflammatory cytokine comprises or consists of IL-1β. More preferably, IL-1β is:
represented by an amino acid sequence comprising at least 60%, 70%, 80%, 90%, 95%, or 100% identity with SEQ ID NO:2 and/or
encoded by a nucleotide acid sequence which has at least 60%, 70%, 80%, 90%, 95%, or 100% identity with SEQ ID NO:1.

In another preferred embodiment, a nucleotide acid sequence encoding IL-1β has:
at least 60%, 70%, 80%, 90%, 95%, or 100% identity with SEQ ID NO:1 and/or
encodes an amino acid sequence of IL-1β that has at least 60%, 70%, 80%, 90%, 95%, or 100% identity with an amino acid sequence encoded by SEQ ID NO:2.

In a preferred embodiment, a pro-inflammatory cytokine comprises or consists of IL-6. More preferably, IL-6 is:
represented by an amino acid sequence comprising at least 60%, 70%, 80%, 90%, 95%, or 100% identity with SEQ ID NO:4 and/or
encoded by a nucleotide acid sequence which has at least 60%, 70%, 80%, 90%, 95%, or 100% identity with SEQ ID NO:3.

In another preferred embodiment, a nucleotide acid sequence encoding IL-6 has:
at least 60%, 70%, 80%, 90%, 95%, or 100% identity with SEQ ID NO:3 and/or
encodes an amino acid sequence of IL-6 that has at least 60%, 70%, 80%, 90%, 95%, or 100% identity with an amino acid sequence encoded by SEQ ID NO:4.

In a preferred embodiment, a pro-inflammatory cytokine comprises or consists of IL-17. More preferably, IL-17 is:
represented by an amino acid sequence comprising at least 60%, 70%, 80%, 90%, 95%, or 100% identity with SEQ ID NO:6 and/or
encoded by a nucleotide acid sequence which has at least 60%, 70%, 80%, 90%, 95%, or 100% identity with SEQ ID NO:5.

In another preferred embodiment, a nucleotide acid sequence encoding IL-17 has:
at least 60%, 70%, 80%, 90%, 95%, or 100% identity with SEQ ID NO:5 and/or
encodes an amino acid sequence of IL-17 that has at least 60%, 70%, 80%, 90%, 95%, or 100% identity with an amino acid sequence encoded by SEQ ID NO:6.

In a preferred embodiment, a pro-inflammatory cytokine comprises or consists of IL-23. More preferably, IL-23 is:
represented by an amino acid sequence comprising at least 60%, 70%, 80%, 90%, 95%, or 100% identity with SEQ ID NO:8 and/or
encoded by a nucleotide acid sequence which has at least 60%, 70%, 80%, 90%, 95%, or 100% identity with SEQ ID NO:7.

In another preferred embodiment, a nucleotide acid sequence encoding IL-23 has:
at least 60%, 70%, 80%, 90%, 95%, or 100% identity with SEQ ID NO:7 and/or
encodes an amino acid sequence of IL-23 that has at least 60%, 70%, 80%, 90%, 95%, or 100% identity with an amino acid sequence encoded by SEQ ID NO:8.

In a preferred embodiment, a pro-inflammatory cytokine comprises or consists of TNFα. More preferably, TNFα is:
represented by an amino acid sequence comprising at least 60%, 70%, 80%, 90%, 95%, or 100% identity with SEQ ID NO:10 and/or
encoded by a nucleotide acid sequence which has at least 60%, 70%, 80%, 90%, 95%, or 100% identity with SEQ ID NO:9.

In another preferred embodiment, a nucleotide acid sequence encoding TNFα has:
at least 60%, 70%, 80%, 90%, 95%, or 100% identity with SEQ ID NO:9 and/or
encodes an amino acid sequence of TNFα that has at least 60%, 70%, 80%, 90%, 95%, or 100% identity with an amino acid sequence encoded by SEQ ID NO:10.

In a preferred embodiment, a pro-inflammatory cytokine comprises or consists of IFNγ. More preferably, IFNγ is:
represented by an amino acid sequence comprising at least 60%, 70%, 80%, 90%, 95%, or 100% identity with SEQ ID NO:12 and/or
encoded by a nucleotide acid sequence which has at least 60%, 70%, 80%, 90%, 95%, or 100% identity with SEQ ID NO:11.

In another preferred embodiment, a nucleotide acid sequence encoding IFNγ has:
at least 60%, 70%, 80%, 90%, 95%, or 100% identity with SEQ ID NO:11 and/or
encodes an amino acid sequence of IFNγ that has at least 60%, 70%, 80%, 90%, 95%, or 100% identity with an amino acid sequence encoded by SEQ ID NO:12.

In a preferred embodiment, a pro-inflammatory cytokine comprises or consists of IL-12. More preferably, IL-12 is:
represented by an amino acid sequence comprising at least 60%, 70%, 80%, 90%, 95%, or 100% identity with SEQ ID NO:26 and/or
encoded by a nucleotide acid sequence which has at least 60%, 70%, 80%, 90%, 95%, or 100% identity with SEQ ID NO:25.

In another preferred embodiment, a nucleotide acid sequence encoding IL-12 has:
at least 60%, 70%, 80%, 90%, 95%, or 100% identity with SEQ ID NO:25 and/or
encodes an amino acid sequence of IL-12 that has at least 60%, 70%, 80%, 90%, 95%, or 100% identity with an amino acid sequence encoded by SEQ ID NO:26.

In a preferred embodiment, a pro-inflammatory cytokine comprises or consists of IL-5. More preferably, IL-5 is:
represented by an amino acid sequence comprising at least 60%, 70%, 80%, 90%, 95%, or 100% identity with SEQ ID NO: 30 and/or
encoded by a nucleotide acid sequence which has at least 60%, 70%, 80%, 90%, 95%, or 100% identity with SEQ ID NO:29.

In another preferred embodiment, a nucleotide acid sequence encoding IL-12 has:
at least 60%, 70%, 80%, 90%, 95%, or 100% identity with SEQ ID NO:29 and/or
encodes an amino acid sequence of IL-12 that has at least 60%, 70%, 80%, 90%, 95%, or 100% identity with an amino acid sequence encoded by SEQ ID NO:30.

Identity is later herein defined. The quantification of the amount of a nucleotide sequence encoding a pro-inflammatory cytokine (preferably IL-1β, IL-6, IL-17, IL-23, IL-12, IL-5, TNFα and/or IFN-γ) is preferably performed using classical molecular biology techniques such as (real time) PCR, arrays or northern analysis. In this embodiment, a nucleotide sequence encoding said pro-inflammatory cytokine (preferably IL-1β, IL-6, IL-17, IL-23, IL-12, IL-5, TNFα and/or IFN-γ) as described herein means a messenger RNA (mRNA). Alternatively, according to another preferred embodiment, in the method the expression level of said pro-inflammatory cytokine (preferably IL-1β, IL-6, IL-17, IL-23, IL-12, IL-5, TNFα and/or IFN-γ) is determined directly by quantifying the amounts of said pro-inflammatory cytokine (preferably IL-1β, IL-6, IL-17, IL-23, IL-12, IL-5, TNFα and/or IFN-γ). Quantifying a polypeptide amount may be carried out by any known technique. Preferably, a polypeptide amount is quantified using a molecule that specifically binds to said pro-inflammatory cytokine (preferably IL-1β, IL-6, IL-17, IL-23, IL-12, IL-5, TNFα and/or IFN-γ). Preferred binding molecules are selected from: an antibody, which has been specifically raised for recognizing said pro-inflammatory cytokine (preferably IL-1β, IL-6, IL-17, IL-23, IL-12, IL-5, TNFα and/or IFN-γ), any other molecule which is known to specifically bind said pro-inflammatory cytokine (preferably IL-1β, IL-6, IL-17, IL-23, IL-12, IL-5, TNFα and/or IFN-γ). Such antibody could be used in any immunoassay known to the skilled person such as western blotting, or ELISA (Enzyme-Linked Immuno Sorbent Assay) or FACS (Fluorescence Activated Cell Sorting) using latex beads. The preparation of an antibody is known to those skilled in the art. Preferably, the presence of a pro-inflammatory cytokine as IL-1β is assessed as carried out in the experimental data. A short explanation of methods that could be used to prepare antibodies is later herein given. In the context of the invention, any other molecule known to bind said pro-inflammatory cytokine (preferably IL-1β, IL-6, IL-17, IL-23, IL-12, IL-5, TNFα and/or IFN-γ) may be a nucleic acid, e.g. a DNA regulatory region, a polypeptide, a metabolite, a substrate, a regulatory element, a structural component, a chaperone (transport) molecule, a peptide mimetic, a non-peptide mimetic, or any other type of ligand. Peptide mimetic is later herein defined. Examples of molecules known to bind said pro-inflammatory cytokine (preferably IL-1β, IL-6, IL-17, IL-23, IL-12, IL-5, TNFα and/or IFN-γ), include a receptor of said pro-inflammatory cytokine (preferably IL-1β, IL-6, IL-17, IL-23, IL-12, IL-5, TNFα and/or IFN-γ) such as the IL-1β receptor, IL-6 receptor, IL-17 receptor, IL-23 receptor, TNFα receptor and/or IFN-γ receptor, an antibody directed against said pro-inflammatory cytokine (preferably IL-1β, IL-6, IL-17, IL-23, IL-12, IL-5, TNFα and/or IFN-γ). Binding of said pro-inflammatory cytokine (preferably IL-1β, IL-6, IL-17, IL-23, IL-12, IL-5, TNFα and/or IFN-γ) to a second binding molecule may be detected by any standard methods known to those skilled in the art. Suitable methods include affinity chromatography co-electrophoresis (ACE) assays and ELISA. The skilled person will understand that alternatively or in combination with the quantification of a nucleic acid sequence encoding said pro-inflammatory cytokine and/or a corresponding polypeptide (preferably IL-1β, IL-6, IL-17, IL-23, IL-12, IL-5, TNFα and/or IFN-γ), the quantification of a substrate of a corresponding polypeptide or of any compound known to be associated with a function or activity of a corresponding polypeptide or the quantification of a function or activity of a corresponding polypeptide using a specific assay is encompassed within the scope of the method of the invention. For example, trans-activation of a target gene by said pro-inflammatory cytokine (preferably IL-1β, IL-6, IL-17, IL-23, IL-12, IL-5, TNFα and/or IFN-γ) or a molecule which is able to bind said pro-inflammatory cytokine (preferably IL-1β, IL-6, IL-17, IL-23, IL-12, IL-5, TNFα and/or IFN-γ) can be determined and quantified, e.g., in a transient transfection assay in which the promoter of the target gene is linked to a reporter gene, e.g., P-galactosidase or luciferase. Such evaluations can be done in vitro or in vivo or ex vivo.

Preferred primers for the detection of a nucleotide sequence encoding a pro-inflammatory cytokine are given below.

Preferred primers used for the IL-1β PCR are identified as

### Forward Primer CAGCTACGAATCTCCGACCAC (SEQ ID NO:13); and

### Reverse Primer GGCAGGGAACCAGCATCTTC (SEQ ID NO:14).

Preferred primers used for the IL-6 PCR are identified as

### Forward Primer AATTCGGTACATCCTCGACGG (SEQ ID NO:15); and

### Reverse Primer GGTTGTTTTCTGCCAGTGCCT (SEQ ID NO:16).

Preferred primers used for the IL-17 PCR are identified as

### Forward Primer TCCCACGAAATCCAGGATGC (SEQ ID NO:17); and

### Reverse Primer GGATGTTCAGGTTGACCATCAC (SEQ ID NO:18).

Preferred primers used for the IL-23 PCR are identified as

### Forward Primer CTCAGGGACAACAGTCAGTTC (SEQ ID NO:19); and

### Reverse Primer ACAGGGCTATCAGGGAGCA-(SEQ ID NO:20).

Preferred primers used for the TNFα PCR are identified as

### Forward Primer TGGCCCAGGCAGTCAGA (SEQ ID NO:21); and

### Reverse Primer GGTTTGCTACAACATGGGCTACA (SEQ ID NO:22).

Preferred primers used for the IFNγPCR are identified as

### Forward Primer TCGGTAACTGACTTGAATGTCCA (SEQ ID NO:23); and

### Reverse Primer TCCTTTTTCGCTTCCCTGTTTT (SEQ ID NO:24).

Preferred primers used for the IL-12 PCR are identified as

### Forward Primer CCTTGCACTTCTGAAGAGATTGA (SEQ ID NO:27); and

### Reverse Primer ACAGGGCCATCATAAAAGAGGT (SEQ ID NO:28).

Preferred primers used for the IL-5 PCR are identified as

### Forward Primer CCTTGCACTTCTGAAGAGATTGA (SEQ ID NO:31); and

### Reverse Primer ACAGGGCCATCATAAAAGAGGT (SEQ ID NO:32).

Optionally in a first method of the invention, one may compare the profile or the expression level of a pro-inflammatory cytokine as determined in step (c) with reference values for said expression levels or profiles, the reference values preferably being the average value for said expression levels or profiles in a control sample.

In the context of the invention, “a reference value” for the profiles or the expression level of said pro-inflammatory cytokine is preferably the average value for said expression levels or profiles in a control sample.

The same holds for the second method, wherein the number of B cells is compared with the number of B cells for a reference, preferable a control sample. In the context of the invention, “a reference value” for the number of B cells is preferably the average number of B cells in a control sample.

Two types of preferred control samples are defined later herein: one for step (b1) and one for step (b2).

In a preferred embodiment of the first method (i.e. for assessing the efficacy of an inhibitor of a pro-inflammatory cytokine), at the end of the contact step (b1) and/or (b2), the supernatant is isolated by centrifugation and the proteins of said pro-inflammatory cytokine (preferably IL-1β, IL-6, IL-17, IL-12, IL-23, IL-5, TNFα and/or IFN-γ) are determined by a skilled person using known methods. The centrifugation may be at 1200 rpm at 4° C. Alternatively, one may add a detergent to the sample at the end of step (b1) and/or (b2). Several detergents could be used such as Triton X 0.1%. Adding a detergent is attractive since it is expected that no centrifugation step is needed. One may determine the expression level of said pro-inflammatory cytokine (preferably IL-1β, IL-6, IL-17, IL-12, IL-23, IL-5, TNFα and/or IFN-γ) in the sample comprising said detergent, which is also called a cell lysate.

In a preferred embodiment of the second method (i.e. for assessing the efficacy of an inhibitor of B cells), at the end of the contact step (b1) and/or (b2), the B cells are isolated by using a specific B cell marker as earlier defined herein.

In step (d) of the first or second method, the efficacy of said inhibitor is assessed.

In the first method, the efficacy of said inhibitor of a pro-inflammatory cytokine is preferably said to be sufficient when at the end of step (b1) a detectable expression level or an increase of the expression level of said pro-inflammatory cytokine has been detected and when at the end of step (b2) a detectable decrease of the expression level of said pro-inflammatory cytokine has been detected.

In the second method, the efficacy of said inhibitor of B cells is preferably said to be sufficient when at the end of step (b1) a detectable number or an increase of the number of said B cells has been detected and when at the end of step (b2) a detectable decrease of the number of said B cells has been detected.

In step (d) following step (b1) of the first method (i.e. for assessing the efficacy of an inhibitor of a pro-inflammatory cytokine), a detectable expression level or profile or an increase of the expression level of said pro-inflammatory cytokine (preferably IL-1β, IL-6, IL-17, IL-23, IL-12, IL-5, TNFα and/or IFN-γ) and/or of their corresponding nucleotide sequences (or steady state levels of said pro-inflammatory cytokine (preferably IL-1β, IL-6, IL-17, IL-23, IL-12, IL-5, TNFα and/or IFN-γ)) or any detectable activities thereof or detectable change in a biological activity thereof) is assessed using a method as defined earlier on and as compared to the expression profile of said pro-inflammatory cytokine (preferably IL-1β, IL-6, IL-17, IL-23, IL-12, IL-5, TNFα and/or IFN-γ) and/or of corresponding nucleotide sequences (or steady state levels of the corresponding encoded pro-inflammatory cytokine (preferably IL-1β, IL-6, IL-17, IL-23, IL-12, IL-5, TNFα and/or IFN-γ) in a control. A preferred control is a similar sample from the same subject, said control sample being not contacted with a compound able to induce the production of a pro-inflammatory cytokine. Usually in said control, the expression level of a pro-inflammatory cytokine is low or undetectable. According to a preferred embodiment, a detection or an increase or a change of activity of said pro-inflammatory cytokine (preferably IL-1β, IL-6, IL-17, IL-23, IL-12, IL-5, TNFα and/or IFN-γ) is quantified using a specific mRNA assay for the genes/nucleotide sequence encoding said pro-inflammatory cytokine (preferably IL-1β, IL-6, IL-17, IL-23, IL-12, IL-5, TNFα and/or IFN-γ).

Preferably, an increase of the expression level of a nucleotide sequence encoding said pro-inflammatory cytokine (preferably IL-1β, IL-6, IL-17, IL-23, IL-12, IL-5, TNFα and/or IFN-γ) means an increase of at least 5% of the expression level of said nucleotide sequence using PCR.

More preferably, an increase of the expression level of a nucleotide sequence means an increase of at least 10%, even more preferably at least 20%, at least 30%, at least 40%, at least 50%, at least 70%, at least 90%, at least 150%, or more.

A low or undetectable profile or expression level of a pro-inflammatory cytokine (preferably IL-1β, IL-6, IL-17, IL-23, IL-12, IL-5, TNFα and/or IFN-γ) preferably means that using PCR, the expression level of a nucleotide sequence encoding said T lymphocyte growth factor (preferably IL-1β, IL-6, IL-17, IL-23, IL-12, IL-5, TNFα and/or IFN-γ) is not detectable, or the Ct value is 35 or higher.

Preferably, an increase of the expression level of said pro-inflammatory cytokine (preferably IL-1β, IL-6, IL-17, IL-23, IL-12, IL-5, TNFα and/or IFN-γ) means an increase of at least 5% of the expression level of said pro-inflammatory cytokine (preferably IL-1β, IL-6, IL-17, IL-23, IL-12, IL-5, TNFα and/or IFN-γ) using western blotting and/or using ELISA or a suitable assay. More preferably, an increase of the expression level of said polypeptide means an increase of at least 10%, even more preferably at least 20%, at least 30%, at least 40%, at least 50%, at least 70%, at least 90%, at least 150%, or more.

Preferably, an increase of an activity of said pro-inflammatory cytokine (preferably IL-1β, IL-6, IL-17, IL-23, IL-12, IL-5, TNFα and/or IFN-γ) means an increase of at least 5% of the polypeptide activity using a suitable assay. More preferably, an increase of the polypeptide activity means an increase of at least 10%, even more preferably at least 20%, at least 30%, at least 40%, at least 50%, at least 70%, at least 90%, at least 150% or more.

In step (d) following step (b2) of the first method (i.e. for assessing the efficacy of an inhibitor of a pro-inflammatory cytokine), a detectable decrease of expression level of said pro-inflammatory cytokine (preferably IL-1β, IL-6, IL-17, IL-23, IL-12, IL-5, TNFα and/or IFN-γ) and/or of their corresponding nucleotide sequences (or steady state levels of said pro-inflammatory cytokine (preferably IL-1β, IL-6, IL-17, IL-23, IL-12, IL-5, TNFα and/or IFN-γ)) or any detectable activities thereof or detectable change in a biological activity thereof) is assessed using a method as defined earlier on and as compared to the expression profile or level of said pro-inflammatory cytokine (preferably IL-1β, IL-6, IL-17, IL-23, IL-12, IL-5, TNFα and/or IFN-γ) and/or of corresponding nucleotide sequences (or steady state levels of the corresponding encoded pro-inflammatory cytokine (preferably IL-1β, IL-6, IL-17, IL-23, IL-12, IL-5, TNFα and/or IFN-γ) in a control. A preferred control is a similar sample from the same subject, said control sample being not contacted with an inhibitor of said pro-inflammatory cytokine. According to a preferred embodiment, a decrease of expression level or a change of activity of said pro-inflammatory cytokine (preferably IL-1β, IL-6, IL-17, IL-23, IL-12, IL-5, TNFα and/or IFN-γ) is quantified using a specific mRNA assays for the genes/nucleotide sequences encoding said pro-inflammatory cytokine (preferably IL-1β, IL-6, IL-17, IL-23, IL-12, IL-5, TNFα and/or IFN-γ).

Preferably, an decrease of the expression level of a nucleotide sequence encoding said pro-inflammatory cytokine (preferably IL-1β, IL-6, IL-17, IL-23, IL-12, IL-5, TNFα and/or IFN-γ) means an decrease of at least 5% of the expression level of said nucleotide sequence using PCR.

More preferably, an decrease of the expression level of a nucleotide sequence means an decrease of at least 10%, even more preferably at least 20%, at least 30%, at least 40%, at least 50%, at least 70%, at least 90%, at least 150%, or more.

Preferably, a decrease of the expression level of said pro-inflammatory cytokine (preferably IL-1β, IL-6, IL-17, IL-23, IL-12, IL-5, TNFα and/or IFN-γ) means a decrease of at least 5% of the expression level of said pro-inflammatory cytokine (preferably IL-1β, IL-6, IL-17, IL-23, IL-12, IL-5, TNFα and/or IFN-γ) using western blotting and/or using ELISA or a suitable assay. More preferably, a decrease of the expression level of said polypeptide means a decrease of at least 10%, even more preferably at least 20%, at least 30%, at least 40%, at least 50%, at least 70%, at least 90%, at least 150%, or more.

Preferably, a decrease of an activity of said pro-inflammatory cytokine (preferably IL-1β, IL-6, IL-17, IL-23, IL-12, IL-5, TNFα and/or IFN-γ) means a decrease of at least 5% of the polypeptide activity using a suitable assay. More preferably, a decrease of the polypeptide activity means a decrease of at least 10%, even more preferably at least 20%, at least 30%, at least 40%, at least 50%, at least 70%, at least 90%, at least 150% or more.

In a preferred method, the efficacy of an inhibitor of a pro-inflammatory cytokine is said sufficient when the expression level of said pro-inflammatory cytokine assessed in step (d) following step (b1) has been increased and the expression level of said pro-inflammatory cytokine assessed in step (d) following step (b2) has been decreased.

In a more preferred method, the efficacy of an inhibitor of a pro-inflammatory cytokine is said sufficient when the expression level of said pro-inflammatory cytokine assessed in step (d) following step (b1) has been increased of at least 20%, 30%, 40%, 50% and the expression level of said pro-inflammatory cytokine assessed in step (d) following step (b2) has been decreased of at least 10%, 20%, 30%, 40%, 50%, 60%, 70%, 80%, 90%. This increase and decrease are preferably assessed using ELISA as earlier described herein.

Even more preferably, the increase assessed in step (d) following step (b1) is of at least 30% and the decrease assessed in step (d) following step (b2) is of at least 30%.

Even more preferably, the increase assessed in step (d) following step (b1) is of at least 40% and the decrease assessed in step (d) following step (b2) is of at least 40%.

Even more preferably, the increase assessed in step (d) following step (b1) is of at least 50% and the decrease assessed in step (d) following step (b2) is of at least 50%.

In step (d) following step (b1) of the second method (i.e. for assessing the efficacy of an inhibitor of B cells), a detectable number or an increase of the number of said B cells has been detected (or any detectable activities thereof or detectable change in a biological activity thereof) is assessed using a method as defined earlier on and as compared to B cells in a control. A preferred control is a similar sample from the same subject, said control sample being not contacted with a compound able to induce the production of B cells. Usually in said control, the number of B cells is low or undetectable. According to a preferred embodiment, B cells are detected using FACS or PCR as earlier explained herein. Alternatively an increase or a change of an activity of a B cell may be the production of a pro-inflammatory cytokine as IL-6 or IL-10 or may be to promote the production of a pro-inflammatory cytokine by other cells, such as T helper cells (Th17). Th17 are known to be able to produce IL-17 as a pro-inflammatory cytokine.

Preferably, an increase of the number of B cells means an increase of at least 5% of the number of B cells, more preferably using FACS or PCR.

More preferably, an increase of the number of B cells means an increase of at least 10%, even more preferably at least 20%, at least 30%, at least 40%, at least 50%, at least 70%, at least 90%, at least 150%, or more.

A low or undetectable number of B cells preferably means that using PCR, no B cells is detected, or the Ct value is 35 or higher.

Preferably, an increase of an activity of a B cell means an increase of at least 5% of the activity using a suitable assay. More preferably, an increase of said activity means an increase of at least 10%, even more preferably at least 20%, at least 30%, at least 40%, at least 50%, at least 70%, at least 90%, at least 150% or more. More preferred activity in this context is the production of IL-6, IL-10 or the production of IL-17 via Thelper17 cells.

Alternatively, an increase of an activity of a B cell means an increase of at least 5% of the number of Th17 cells, more preferably using FACS or PCR.

More preferably, an increase of the number of Th17 cells means an increase of at least 10%, even more preferably at least 20%, at least 30%, at least 40%, at least 50%, at least 70%, at least 90%, at least 150%, or more.

In step (d) following step (b2) of the second method (i.e. for assessing the efficacy of an inhibitor of B cells), a detectable decrease of the number of said B cells has been detected and/or of any detectable activities thereof or detectable change in a biological activity thereof) is assessed using a method as defined earlier on and as compared to the number of B cells and/or of corresponding activity in a control. A preferred control is a similar sample from the same subject, said control sample being not contacted with an inhibitor of said B cells. According to a preferred embodiment, a decrease of the number of B cells or a change of activity of said B cells is quantified as identified earlier herein.

Preferably, a decrease of the number of B cells means a decrease of at least 5% of the expression level of said nucleotide sequence using FACS.

More preferably, a decrease of the number of B cells means a decrease of at least 10%, even more preferably at least 20%, at least 30%, at least 40%, at least 50%, at least 70%, at least 90%, at least 150%, or more.

Preferably, a decrease of an activity of said B cells means a decrease of at least 5% of said activity using a suitable assay. More preferably, a decrease of said activity means a decrease of at least 10%, even more preferably at least 20%, at least 30%, at least 40%, at least 50%, at least 70%, at least 90%, at least 150% or more.

More preferred activity in this context is the production of IL-6, IL-10 or the production of IL-17 via Thelper17 cells.

Alternatively, a decrease of an activity of a B cell means a decrease of at least 5% of the number of Th17 cells, more preferably using FACS or PCR.

More preferably, a decrease of the number of Th17 cells means a decrease of at least 10%, even more preferably at least 20%, at least 30%, at least 40%, at least 50%, at least 70%, at least 90%, at least 150%, or more.

In a preferred second method, the efficacy of an inhibitor of B cells is said sufficient when the number of B cells assessed in step (d) following step (b1) has been increased and the number of B cells assessed in step (d) following step (b2) has been decreased.

In a more preferred second method, the efficacy of an inhibitor of B cells is said sufficient when the number of B cells assessed in step (d) following step (b1) has been increased of at least 20%, 30%, 40%, 50% and the number of B cells assessed in step (d) following step (b2) has been decreased of at least 10%, 20%, 30%, 40%, 50%, 60%, 70%, 80%, 90%. This increase and decrease are preferably assessed using FACS or PCR as earlier described herein.

Even more preferably, the increase assessed in step (d) following step (b1) is of at least 30% and the decrease assessed in step (d) following step (b2) is of at least 30%.

Even more preferably, the increase assessed in step (d) following step (b1) is of at least 40% and the decrease assessed in step (d) following step (b2) is of at least 40%.

Even more preferably, the increase assessed in step (d) following step (b1) is of at least 50% and the decrease assessed in step (d) following step (b2) is of at least 50%.

In another preferred second method, the efficacy of an inhibitor of B cells is said sufficient when an activity of B cells assessed in step (d) following step (b1) has been increased and an activity of B cells assessed in step (d) following step (b2) has been decreased.

In a more preferred second method, the efficacy of an inhibitor of B cells is said sufficient when an activity of B cells assessed in step (d) following step (b1) has been increased of at least 20%, 30%, 40%, 50% and an activity of B cells assessed in step (d) following step (b2) has been decreased of at least 10%, 20%, 30%, 40%, 50%, 60%, 70%, 80%, 90%. This increase and decrease are preferably assessed using FACS or PCR or ELISA as earlier described herein.

Even more preferably, the increase assessed in step (d) following step (b1) is of at least 30% and the decrease assessed in step (d) following step (b2) is of at least 30%.

Even more preferably, the increase assessed in step (d) following step (b1) is of at least 40% and the decrease assessed in step (d) following step (b2) is of at least 40%.

Even more preferably, the increase assessed in step (d) following step (b1) is of at least 50% and the decrease assessed in step (d) following step (b2) is of at least 50%.

This approach to assess the efficacy of an inhibitor of a pro-inflammatory cytokine and/or to assess the efficacy of an inhibitor of B cells in a subject suspected to have an inflammatory and/or auto-immune condition or disease may be applicable for all treatments based on biological therapies that are currently used. This approach may be of great importance for both the patients that would be spared treatments that are unlikely to be successful and that have potential important side-effects, and for the healthcare system due to the substantial cost-saving aspect of such a personalized approach.

### Method for Treating

In a further aspect, the invention relates to a method for treating a subject suspected to suffer from an autoimmune and/or inflammatory condition or disease comprising the following steps of assessing the efficacy of an inhibitor of a pro-inflammatory cytokine and/or assessing the efficacy of an inhibitor of B cells in a subject as defined earlier herein and subsequently if the efficacy of said inhibitor is satisfying, treating said subject with said inhibitor.

The method of assessing the efficacy of an inhibitor of a pro-inflammatory cytokine and/or of assessing the efficacy of an inhibitor of B cells has been extensively explained in the section dedicated to the first aspect of the invention.

A treatment against an autoimmune and/or inflammatory disease or condition may be a long-term administration of one of the inhibitors of a pro-inflammatory cytokine and/or of one of the inhibitors of B cells mentioned earlier herein.

Such a treatment is intended to cure or chronically suppress or alleviate a symptom or a parameter of said subject after at least one week, one month, six month of treatment.

Such a parameter could be the expression level or profile of a pro-inflammatory cytokine and/or the number of B cells as defined earlier herein. Such expression level or profile may normalize towards a lower value than the value measured in said subject at the onset of the treatment. In this context, “lower than” may mean 5% lower than, 10%, 20%, 30%, 40%, 50%, 60%, 70%, 80%, 90% or 100% lower than or more.

### Sequence Identity

“Sequence identity” is herein defined as a relationship between two or more amino acid (polypeptide or protein) sequences or two or more nucleic acid (polynucleotide) sequences, as determined by comparing the sequences. The identity between two amino acid or two nucleic acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg, Gln or Glu; Met to Leu or Ile; Phe to Met, Leu or Tyr; Ser to Thr; Thr to Ser; Trp to Tyr; Tyr to Trp or Phe; and Val to Ile or Leu.

### Antibodies

Some aspects of the invention concern the use of antibodies or antibody-fragments that specifically bind to a pro-inflammatory cytokine. Methods for generating antibodies or antibody-fragments that specifically bind to such polypeptides are described in e.g. Harlow and Lane (1988, Antibodies: A Laboratory Manual, Cold Spring Harbor Laboratory Press, Cold Spring Harbor, N.Y.) and WO 91/19818; WO 91/18989; WO 92/01047; WO 92/06204; WO 92/18619; and U.S. Pat. No. 6,420,113 and references cited therein. The term “specific binding,” as used herein, includes both low and high affinity specific binding. Specific binding can be exhibited, e.g., by a low affinity antibody or antibody-fragment having a Kd of at least about 10⁻⁴ M. Specific binding also can be exhibited by a high affinity antibody or antibody-fragment, for example, an antibody or antibody-fragment having a Kd of at least about of 10⁻⁷ M, at least about 10⁻⁸ M, at least about 10⁻⁹ M, at least about 10⁻¹⁰ M, or can have a Kd of at least about 10⁻¹¹ M or 10⁻¹² M or greater.

### Peptidomimetics

A peptide-like molecule (referred to as peptidomimetics) or non-peptide molecule that specifically binds to a pro-inflammatory cytokine as defined herein or to its receptor polypeptide and that may be applied in a method of the invention as defined herein (for assessing the expression level of a pro-inflammatory cytokine) and may be identified using a method known in the art per se, as e.g. described in detail in U.S. Pat. No. 6,180,084 which incorporated herein by reference. Such a methods includes e.g. screening libraries of peptidomimetics, peptides, DNA or cDNA expression libraries, combinatorial chemistry and, particularly useful, phage display libraries. These libraries may be screened for such peptidomimetics of a pro-inflammatory cytokine by contacting the libraries with a substantially purified pro-inflammatory cytokine, fragments thereof or structural analogues thereof.

### General

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a method as defined herein may comprise additional step(s) than the ones specifically identified, said additional step(s) not altering the unique characteristic of the invention. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

All patent and literature references cited in the present specification are hereby incorporated by reference in their entirety. The following examples are offered for illustrative purposes only, and are not intended to limit the scope of the present invention in any way.

### FIGURE LEGENDS

FIG. 1. The effect of a caspase-1 inhibitor (VRT) on IL-1β production. Human PBMCs were exposed to several PRR agonists for 24 h to stimulate the IL-1β production. In addition to PRR ligands, PBMCs were stimulated with PRR ligands in the presence of a caspase-1 inhibitor (Upper left panel). PBMC's of 50 subjects were included in these experiments. In separate panels, the different PPR agonists with or without caspase-1 inhibitor is shown. IL-1β was determined by Elisa.

FIG. 2. The effect of caspase-1 inhibitor (VRT) depending on ATG16L1 genotype. The 50 subjects were genotyped for the ATG16L1 genotype and thereafter the IL-1β production was expressed per genotype. Three different PRR ligands were shown, MDP, LPS and the combination of MDP and LPS. IL-1β was determined by Elisa. Left panels; the IL-1β production of each subject is shown. Right panels; Box-plots are shown.

FIG. 3. The effect of caspase-1 inhibitor (VRT) depending on LPS- and MDP-induced IL-1β production. Stratification of the subjects in high (>2000 pg·ml), moderate (1000-2000 pg/ml) and low (<1000 pg·ml) IL-1β producers after stimulation with LPS or MDP. The effect of caspase-1 inhibition is shown in each group of subjects in percentage inhibition, compared to the vehicle control.

FIG. 4. TNFα production in healthy individuals. PBMCs from 104 individuals were isolated according standard protocols. TNFα production capacity was determined by 24 h exposure to 10 ng/ml E-coli LPS or 10⁶ HK Candida/ml. Thereafter TNFα was measured using ELISA.

FIG. 5. Correlation of the LPS- and Candida-induced TNFα production. From 104 subjects, PBMCs were stimulated with E. coli LPS or HK Candida albicans. TNFα production capacity was determined by 24 h exposure to 10 ng/ml E-coli LPS or 10⁶ HK Candida albicans/ml. Thereafter TNFα was determined by ELISA. The figure showed that not all subjects showed to be high TNFα producer for both LPS and Candida.

FIG. 6. Stratification of Crohn's patients. From 23 IBD patients, PBMCs were isolated and stimulated for 24 h with E. coli LPS (10 ng/ml) or Pam3cys/MDP (10 ug/ml and 10 ug/ml). Thereafter TNFα was determined by ELISA. MDP (Muramyl-Di-Peptide) is seen as a disease specific stimulus since this will be recognized by the intracellular NOD2 receptor.

FIG. 7. PBMC's from 2 RA patients were stimulated with IgG control (IvIg) or 3 different TNFα inhibitors for 30 minutes. Thereafter 10⁶ HK Candida/ml were added. After 24 h, the IL-1β production was measured by ELISA. Anti-TNFα were tested in a dose of 4 ug/ml, which is the dose that will be present in a RA patients after anti-TNFα therapy. Note the all 3 TNFα blockers reduced the IL-1β production. It is known that TNFα contributes to the IL-1β production by PBMCs elicited by Candida exposure.

FIG. 8. PBMCs of 188 gout patients were stimulated for 24 h with Medium (RPMI1640) Pam3Cys, or MSU/C16.0. Thereafter, IL-1β was measured using ELISA. Here it is seen that MSU/C16.0 (gout specific) is a potent inducer of IL-1β.

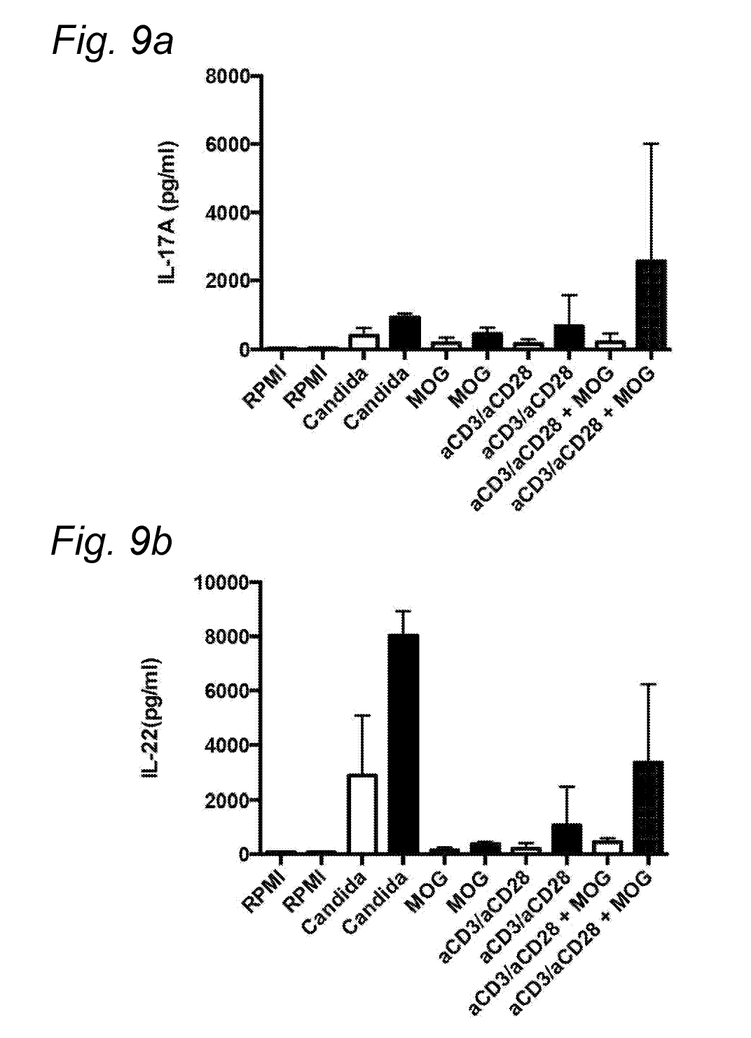

FIG. 9. Stratification of MS patients. PBMCs isolated from 4 MS patients and 4 age and sex matched healthy controls were stimulated for 7 days with RPMI, Candida albicans (1.10⁶/ml), MOG peptide (10 μg/ml), Anti-CD3/CD28 (1 μg/ml:0.1 μg/ml) and the combination of MOG/Anti-CD3/28 (10 μg/ml and 1 μg/ml:0.1 μg/ml). FIG. 9A showed that the IL-17A production of MS patients was strongly upregulated after exposure to MOG/Anti-CD3/28 when compared to healthy controls. FIG. 9B showed that the IL-22 production by PBMCs is elevated in MS patients after exposure to Candida albicans and MOG/Anti-CD3/28 when compared to controls. Interestingly, the IL-22 production after MOG/Anti-CD3/28 stimulation is strongly upregulated. No major differences were noted in the IFN-γ production between and MS patients and healthy controls, although the Candida albicans induced IFN-g is slightly higher (FIG. 9C).

TABLE 1
Stratification of inflammatory diseases based on cytokine/B cells involvement.

| | | | Other | | | | | | | | | |
|---|---|---|---|---|---|---|---|---|---|---|---|---|
| | | | RA-like | Ulcerative | Chron's | | | | | | Lyme | Type II |
| Cytokine | Target | RA | diseases | colitis | disease | Psoriasis | MS | Asthma | Sepsis | Gout | disease | Diabetes |
| TNF [symbol] | | X | X | X | X | X | | | | | | X |
| IL-1 [symbol] | | X | | | X | | X | | X | X | X | X |
| IL-5 | | | | | | | | X | | | | |
| IL-6 | IL-6R | X | | | | | | | | | | |
| IL-12 | IL-12p40 | X | | | X | X | | | | | | |
| IL-17 | IL-17A | X | | | X | X | X | | | | X | |
| IL-17 | IL-17F | | | | | X | X | | | | | |
| IL-23 | | X | | | X | X | | | | | | |
| IFN [symbol] | | | | | | | | X | X | | | |
| B cell | CD20 | X | | | | | X | | | | | |
| | CD19 | X | | | | | X | | | | | |

TABLE 2
known inhibitor of pro-inflammatory cytokine or of B cells

| | |
|---|---|
| TNFα inhibitors | Embrel (Amgen and Whyett, USA); Humira |
| | (Abbott, USA); Infliximab (Centocor |
| | Pharmaceuticals (Johnson&Johnson), |
| | USA); Golimumab (MSD) |
| IL-1β inhibitors | Anakinra (IL-IRa) (Sobi, SE); Ilaris |
| | (Anti-IL-1b) (Novartis, CH) |
| IL-5 inhibitors | Mepolizumab (GSK, UK) |
| IL-6R inhibitors | Tocilizumab (Roche, CH) |
| IL-12p40 | Ustekinumab (Janssen-Cilag, BE) |
| inhibitors | |
| IL-17A inhibitors | Brodalumab (Amgen, USA); Ixekizumab |
| | (;Lilly (Eli), USA) Secukinumab |
| | (Novartis, CH) |
| IL-23 inhibitors | Ustekinumab (Janssen-Cilag, BE) |
| IFNγ□ | Immukine (Boehringer Ingelheim, DE) |
| B-cell (CD20) | Rituximab (Roche, CH), ofatumumab (GSK, |
| | UK), veltuzumab (Takeda, JP) or |
| | ocrelizumab (Roche CH) |
| Bcell (CD19) | GBR 401 (Glenmark Pharmaceuticals, CH). |

### EXAMPLES

### Example 1

### Materials and Methods

Isolation of Human Peripheral Blood Mononuclear Cells and In-Vitro Cytokine Production.

Venous blood was drawn from the cubital vein of healthy volunteers or patients with CGD into 10 ml EDTA tubes (Monoject, Covidien, Mansfield, Mass., USA). The mononuclear cell fraction was obtained by density centrifugation of blood diluted 1:1 in pyrogen-free saline over Ficoll-Paque (Pharmacia Biotech, Pittsburgh, Pa., USA). Cells were washed twice in saline and suspended in culture medium (RPMI; Invitrogen, Carlsbad, Calif., USA) supplemented with gentamicin 10 mg/ml, L-glutamine 10 mM and pyruvate 10 mM. Cells were counted in a Coulter counter (Coulter Electronics, Brea, Calif., USA) and the number was adjusted to 5×10⁶ cells/ml.

A total of 5×10⁵ mononuclear cells in a 100 μl volume was added to round-bottom 96-well plates (Greiner, Monroe, N.C., USA) and incubated with either 100 μl of culture medium (negative control), or LPS (10 ng/ml, Sigma, MO, USA), Pam3Cys (10 μg/ml, EMC Microcollections, Tubingen, Germany), flagellin (TLR5 ligand), MDP (10 μg/ml, Sigma. MO, USA). After 2.4 hours, supernatants were collected and stored at −20° C. until being assayed. PBMCs from a group of 50 healthy donors were stimulated with a panel of well-defined pattern recognition receptor (PRR) ligands. Donor blood was obtained from Sanquin Blood bank, Nijmegen, The Netherlands. IL-1β was measured after 24 hours incubation, using a commercial ELISA kit (R&D Systems, MN, USA).

Genotyping for ATG16L1 Thr300Ala Polymorphism.

DNA was isolated from whole blood by using the isolation kit Puregene (Gentra Sytems, MN, USA), according to the manufacturers' protocol. Genotyping for the presence of the ATG16L1 Thr300Ala polymorphism was performed by applying the TaqMan single nucleotide polymorphism (SNP) assay C_9095577_20 on the 7300 ABI Real-Time polymerase chain reaction system (Applied Biosystems, CA, USA).

### Results

Ex-Vivo Studies in Primary Human Cells.

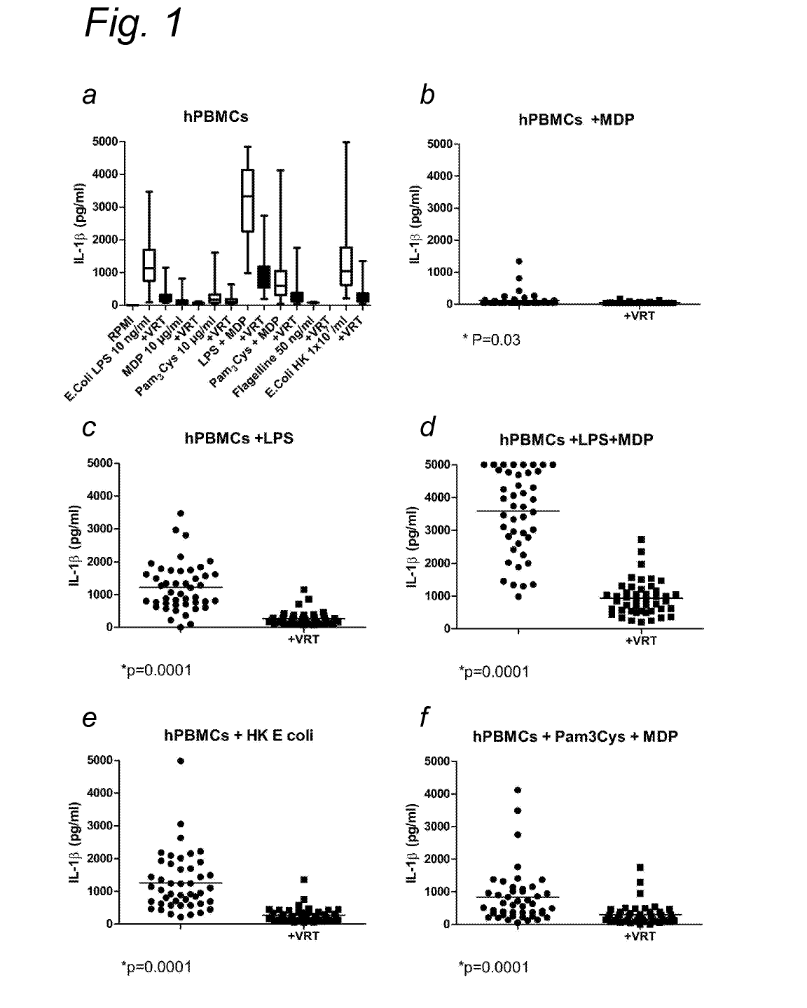

The ability of caspase-1 inhibitors to decrease IL-1β production has been compared in subjects. PBMCs from a group of 50 healthy blood donors was stimulated with well-defined set of pattern recognition receptor ligands that are relevant for intra-intestinal inflammation (e.g. LPS-TLR4 ligand, Pam3Cys-TLR2 ligand, flagellin-TLR5 ligand, MDP-NOD2 ligand, and several combinations of these ligands). FIG. 1A showed that LPS, Pam3cys and MDP are strong inducers of IL-1β production by human PBMCs. Flagellin (TLR5 ligand) was found to be a weak inducer of IL-1β production. When LPS or Pam3cys was combined with MDP a strong up-regulation of IL-1β production was noted (FIG. 1A). Heat-killed E. coli revealed to be a potent inducer of IL-1β, probably due to the multiple TLR/NLR ligands present on the whole microorganisms.

The caspase-1 inhibitor (VRT) displayed significant inhibitory effects on the IL-1β stimulation by a large array of stimuli, including purified TLR ligands, muramyl dipeptide component of peptidoglycans (NOD2 agonist), and whole Gram-negative intestinal bacteria such as Escherichia coli (FIG. 1). Although MDP is a weak inducer of IL-1β, caspase-1 inhibition still significantly (p<0.03) reduced the IL-1β production. Interestingly, when the IL-1β production was strongly elevated by LPS, LPS/MDP, Pam3cys/MDP or HK E. coli the inhibition of the IL-1β production by caspase-1 inhibitor (VRT) was more pronounced (FIG. 1).

The Effect of the Caspase-1 Inhibitor Depending on the ATG16L1 Genotype.

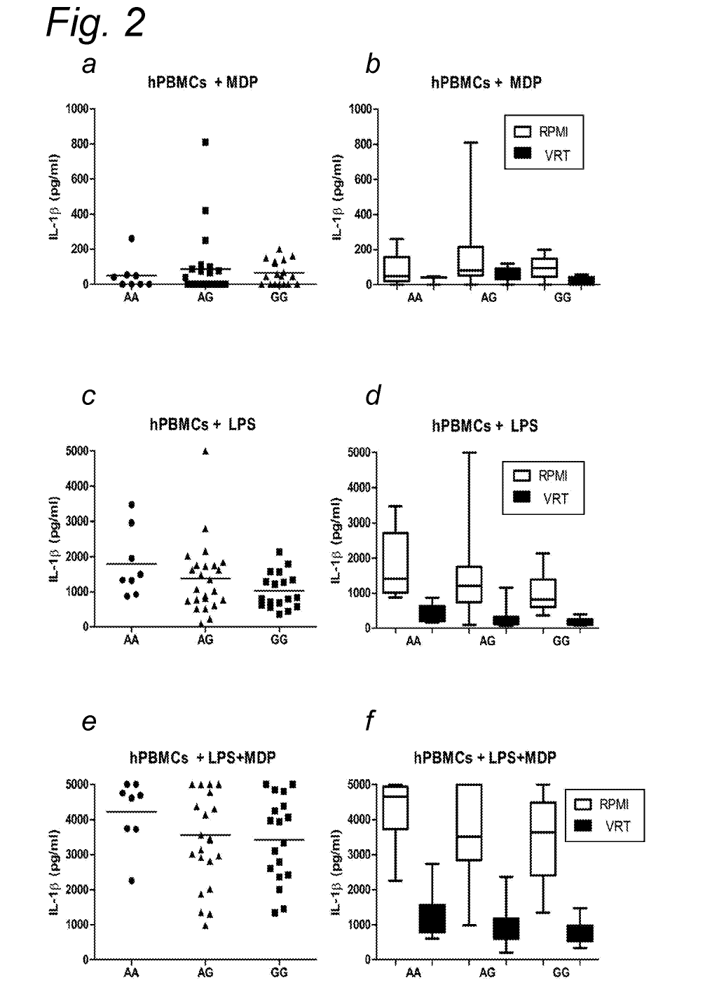

Due to the fact that enhanced IL-1β production was linked to non-functional autophagy machinery were performed a next set of analyses encompassed the assessment whether the effect of the caspase-1 inhibitor (VRT) was dependent on the ATG16L1 genotype. There was no difference in IL-1β production after stimulation with the several ligands with respect to ATG16L1 genotype (FIG. 2). The caspase-1 inhibitor (VRT) was equally effective in all subgroups of individuals, independently of their genotype in the autophagy gene ATG16L1 (FIG. 2).

The Effect of the Caspase-1 Inhibitor Depends on the IL-1β Production Capacity.

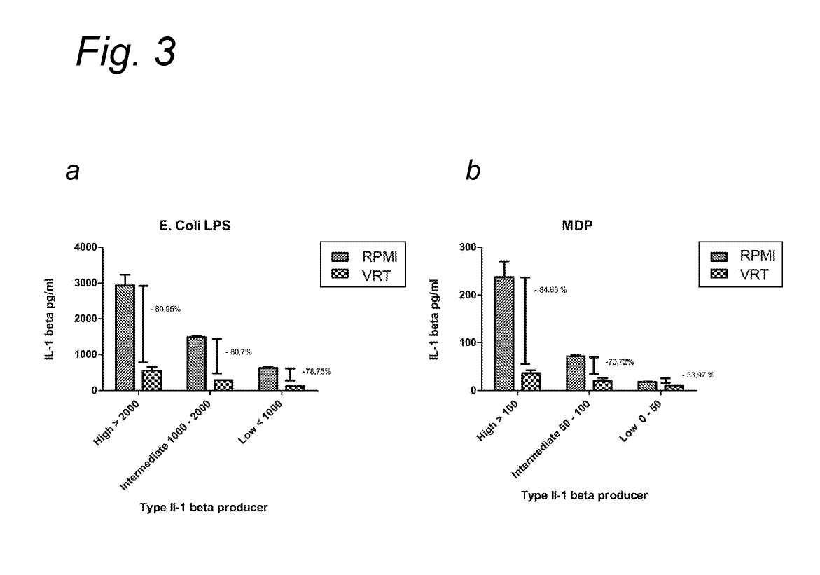

In a next set of experiments, we have assessed the concept that the effect of the caspase-1 inhibitor (VRT) may be dependent on the capacity of individuals to produce high, moderate, or low IL-1β amounts. As shown in FIG. 3, while the caspase-1 inhibitor (VRT) inhibited more than 80% of the IL-1β production induced by LPS, the inhibition of the IL-1β induced by the Crohn-specific stimulus MDP was strongly dependent on the initial production: 85% inhibition in high-producers, 70% inhibition in low-producers, and only 34% inhibition in the low producers.

### Discussion

The caspase-1 inhibitor (VRT) is a strong inhibitor of IL-1β production that holds promise for the future treatment of IL-1β-dependent diseases such as Crohn's disease. This particular caspase-1 inhibitor was very potent for ex-vivo inhibition of IL-1β in human primary cells.

In order to identify the patients most likely to benefit from the therapy with a caspase-1 inhibitor, we have investigated its capacity to inhibit IL-1β in individuals with various T300A ATG16L1 genotypes (4). The caspase-1 inhibitor used in this study (VRT) was able to inhibit IL-1β very potently independently of the T300A ATG16L1 polymorphism. The difference in IL-1β release between the various genotypes, as reported previously (4), was not reproduced in the present study. The reason for this is unclear, although the different MDP batches used in these studies may be one of the reasons. Additional experiments in which MDP was mixed with low amounts of LPS to mimic a possible contamination of commercial products did not provide differences in IL-1β production between genotypes either (not shown).

A second approach for personalized medicine was to assess whether the caspase-1 inhibitor (VRT) was equally effective in individuals displaying a high, moderate, or low capacity to produce IL-1β in response to non-specific stimulation with LPS, or with the NOD2 agonist MDP, which is a stimulus particularly relevant for Crohn's disease. Interestingly, while the caspase-1 inhibitor (VRT) strongly inhibited IL-1β production induced by the potent non-specific stimulus LPS, the inhibition of the IL-1β induced by the Crohn-specific stimulus MDP was strongly dependent on the initial production: 85% inhibition induced by caspase-1 inhibitor in high-producers, 70% inhibition in low-producers, and only 34% inhibition in the low producers. This strongly suggests that the caspase-1 inhibitor may be most effective in high- and moderate-responder individuals releasing high amounts of IL-1β upon stimulation with MDP. A personalized treatment approach with caspase-1 inhibitors, based on the IL-1β-producing status of the individual patients, seems therefore to hold future promise in the treatment of Crohn's disease.

This approach to stratify patient cohorts with (auto)inflammatory diseases in high and low cytokine producers may be applicable for all treatments based on biological therapies that are currently used. The stratification of patients may be of great importance for both the patients that would be spared treatments that are unlikely to be successful and that have potential important side-effects, and for the healthcare system due to the substantial cost-saving aspect of such a personalized approach.

### Example 2

### Materials and Methods

Stratification of healthy individuals. PBMC's of 104 individuals were isolated as described in example 1. A total of 5×10⁵ mononuclear cells in a 100 μl volume was added to round-bottom 96-well plates (Greiner, Monroe, N.C., USA) and incubated with either 100 μl of culture medium (negative control), or LPS (10 ng/ml, Sigma, MO, USA), or HK Candida albicans. (10⁶/ml). After 24 hours, supernatants were collected and stored at −20° C. until being assayed. TNFα was measured after 24 hours incubation, using a commercial ELISA kit (R&D Systems, MN, USA).

### Results

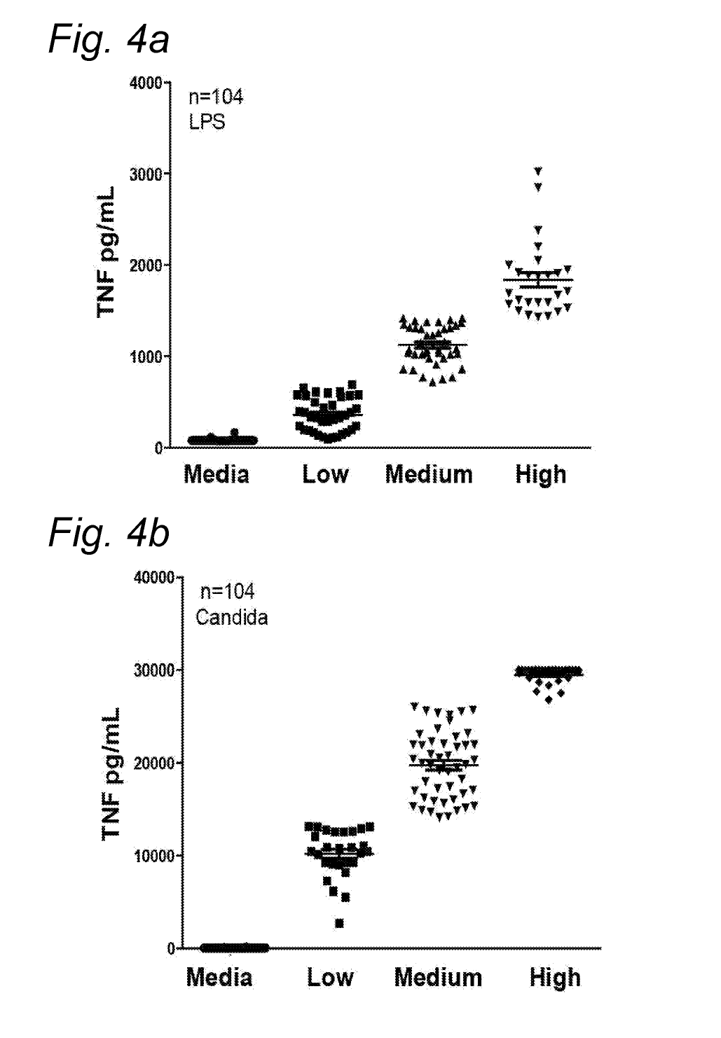

The TNFα production of each individual was evaluated. Based on the intrinsic capacity to produce TNFα the healthy individuals were classified as Low, Medium and High TNFα producers (see FIG. 4). A number of 37 individuals were classified as low TNFα producers, 40 as medium and 27 as high producers after the PBMCs were exposed to LPS (FIG. 4a). The mean TNFα production as each category was respectively, 363 ng/ml, 1120 pg/ml and 1838 pg/ml. FIG. 4b showed the TNFα production of human PBMCs after 24 h exposure to heat killed (HK) Candida albicans. Individuals were classified in similar groups as for LPS stimulation. The mean TNFa production as each category was respectively, 10.182 ng/ml, 19.761 pg/ml and 29.524 pg/ml.

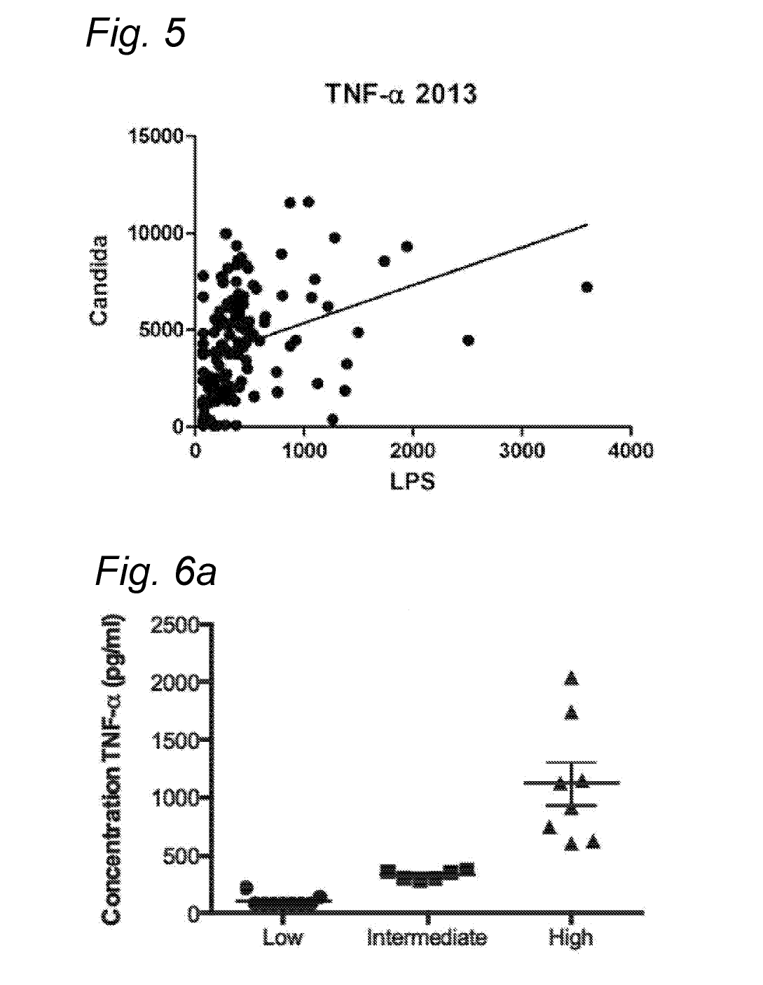

To examine whether a subject produce always a high concentration of TNFα independent on the stimulus the LPS- and Candida albicans-induced TNFα production was correlated. FIG. 5 showed that the R was 0.5111 indicating that not all subjects produce high TNFα after exposure to either LPS or HK Candida albicans.

### Discussion

Using a large numbers of individuals it revealed that each subject can be stratified into low, medium and high producer based on the TNFα production. Of interest, it can be seen that not always an individual produce high TNFα after exposure to Candida albicans or LPS.

### Example 3

### Materials and Methods

Stratification of Crohn's patients. 24 patients with Crohn's disease were screened for their cytokine profile (TNFα). PBMC were isolated as described in example 1. The PBMC were exposed to disease relevant stimuli. LPS (10 ng/ml), Pam3cys (10 μg/ml), Muramyl dipeptide (MDP)(10 μg/ml) and Pam3cys/MDP. MDP is a well-known NOD2 ligand and is regarded as disease specific.

### Results

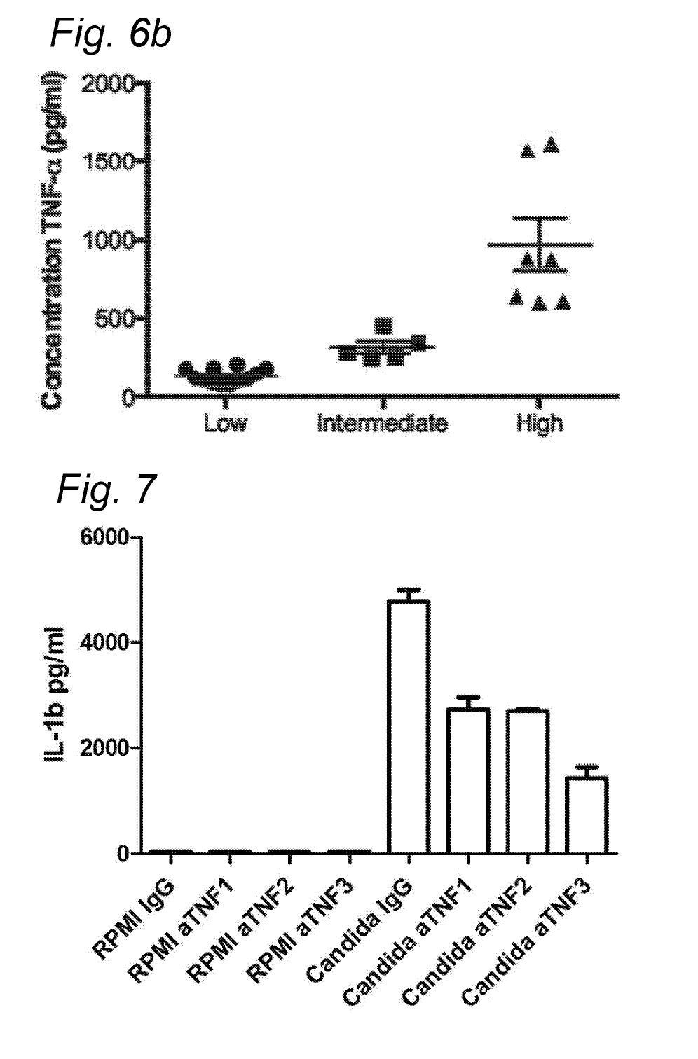

Based on the capacity to produce TNFα upon LPS exposure □□ IBD patients were classified as Low (below 250 pg/ml), Medium (between >250 and <500 pg/ml) and High (>500 pg/ml) TNFα producers. FIG. 6a showed the TNFα production of PBMCs after exposure to LPS. Dependent of the concentrations of TNFα the IBD patients were classified as low, medium and high TNFα producers. FIG. 6b showed the TNFα production of PBMCs after exposure to Pam3Cys/MDP. Similar as for LPS the IBD patients could be divided in 3 groups. Low (below 250 pg/ml), Medium (between >250 and <500 pg/ml) and High (>500 pg/ml).

### Discussion.

Using a large numbers of individual IBD patients it revealed that each subject produce different concentration of TNFα and thereby could be stratified into low, medium and high producers. LPS is assumed to only activate TLR4 while MDP/Pam3Cys is supposed to activate both TLR2 and NOD2 pathways. TLR2 and NOD2 pathways showed a strong synergism. Both pathways are linked to the disease.

Interestingly, the disease-linked trigger Pam3cys/MDP can be used for stratification of the IBD patients. As seen for RA patients, the TNFα production of an individual after exposure to LPS is not highly correlated with the TNFα production after Pam3cys stimulation.

### Example 4

### Material and Methods

Stratification of RA patients. RA patients were screened for basic cytokine profile before starting treatment with a biological. PBMCs were isolated as indicated in example 1. PBMCs were stimulated with a range of stimuli, including LPS, Pam3cys, and Candida albicans. In addition, we added several biologicals (4 μg/ml of Humira, Etanercept or Golumimab, all from Sanquin, The Netherlands, IgIV (Nanogam)) to the culture system to investigate the effect of the particular biological to the ex-vivo cytokine production. PBMC's from RA patients were incubated with IgG control (IvIg) or 3 different TNFα inhibitors as identified above for 30 minutes. Thereafter 10⁶ HK Candida/ml were added. After 24 h, the IL-1β production was measured by ELISA. Anti-TNFα were tested in a dose of 4 μg/ml, which is the dose that will be present in a RA patients after anti-TNFα therapy.

### Results

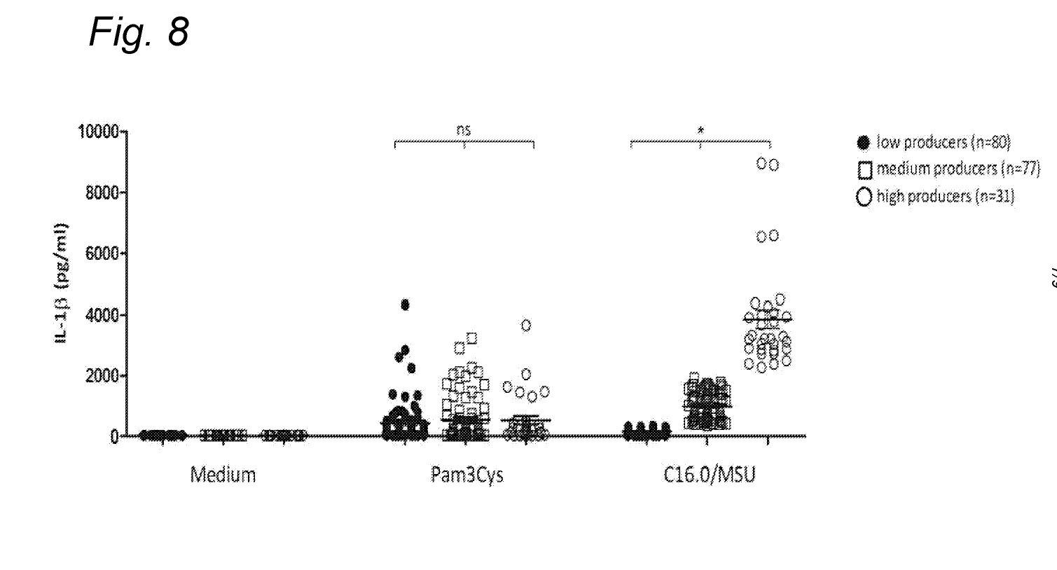

As shown in FIG. 7, PBMC's from RA patients could be stratified based on their production of IL-1β. Note the all 3 TNFα blockers reduced the IL-1β production. It is known that TNFα contributes to the IL-1β production by PBMCs elicited by Candida albicans exposure.

### Discussion

TNFα contributes to the IL-1β production by immune cells after stimulation with HK Candida albicans based on the TNFα production. By using neutralizing TNFα strategies the bioactivity of the ex-vivo produce TNFα could be modulated.

Interestingly, the first results indicated that there is a difference in the neutralizing capacity of the tested anti-TNF modalities.

### Example 5

### Material and Methods

Stratification of MS patients. PBMCs were isolated as described in example 1. PBMC's from the MS patients and age/sex controls were exposed to Candida albicans (1.10⁶/ml), anti-CD3/CD28 (1 μg/ml, 0.1 μg/ml), MOG (MS related peptide, 10 μg/ml) and the combination of MOG/anti-CD3/CD28 for 7 days. Cytokines were measured after day 7. IL-17A, IL-22 and IFN-γ were measured by Elisa.

### Results

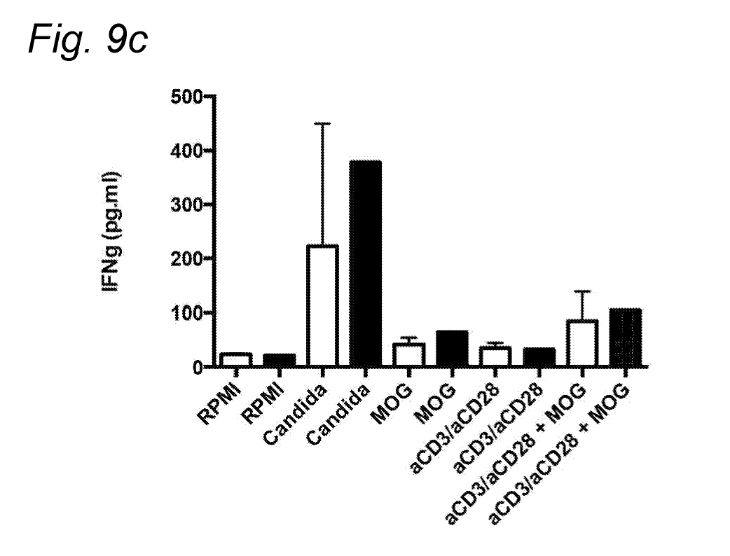

FIG. 9 showed that PBMCs isolated from MS patients produce more IL-17A compared to controls after stimulation with Candida albicans. When PBMCs were exposed to MOG peptide or anti-CD3/CD28 alone an enhanced IL-17A was noted. However, after exposure to anti-CD3/CD28 and MOG peptide MS patients produce strongly enhanced IL-17A concentrations. In line with IL-17A, the concentration of IL-22 (a Th17 related cytokine) revealed to be enhanced in PBMCs from MS patients when exposed to anti-CD3/CD28/MOG peptide. Of interest, the IFNγ production was similar between PBMCs from MS patients and healthy individuals when exposed to MOG peptide, anti-CD3/CD28 or the combination of these two stimuli.

### Discussion

The results showed that PBMCs of MS patients clearly respond differently to disease specific stimuli (MOG peptide and anti-CD3/CD28/MOG peptide). IL-17 and IL-22 production by PBMCs can be used for stratification of MS patients, in contrast to IFN-γ

### Example 6

### Material and Methods

Stratification of Gout patients. 188 Gout patients were analyzed for their intrinsic cytokine production capacity. Since Gout is an IL-1 disease we determined the IL-1β production of PBMCs after exposure to a disease specific stimulus (Mono Sodium Urate (MSU) crystals and fatty acids (C16.0)). MSU was prepared in our laboratory according to techniques known to the skilled person. C16.0 was purchased from Sigma Aldrich (USA). PBMCs were exposed for 24 h to MSU/C16.0 (300 μg/ml, 200 μM C16.0) or Pam3cys (10 μg/ml). Thereafter IL-1β was determined by ELISA.

### Results

As shown in FIG. 9, PBMC's from gout patients could be stratified based on their production of IL-1β after exposure to MSU/C16.0. It has been shown that the combination of MSU and C16.0 (Palmitic acid) is essential for the production of IL-1β. (10). MSU alone does not stimulate the release of IL-1β, neither does C16.0. Of high interest, no synergy of MSU/C16.0 was found for the production of TNFα by human PBMCs. In total a group of 188 gout patients were stratified into low (<350 pg/ml), medium (>3 50<2000 pg/ml) and high (>2000 pg/ml) IL-1β producers. When the IL-1β production, after Pam3cys exposure of the PBMCs, was used for classification of the gout patients it reveals that the Pam3cys-induced IL-1β production was not associated with the MSU/C16.0 production. This latter indicated that MSU/C16.0 can be used as a disease specific stimulus for gout patients for stratification.

### Discussion

The results showed that PBMCs of gout patients clearly respond differently to a disease specific stimulus (MSU/C16.0). The concentrations of IL-1β, the classical cytokine involved in gout was enhanced in gout patients as shown previously (9). The production of IL-1β by PBMCs can be used for stratification of gout patients.

### Example 7

FIG. 5 is made from the data of FIGS. 4A and 4B. From 104 subjects, PBMCs were stimulated with E. coli LPS or HK Candida albicans. TNFα production capacity was determined by 24 h exposure to 10 ng/ml E-coli LPS or 10⁶ HK Candida albicans/ml.

Thereafter TNFα was determined by ELISA. The figure showed that not all subjects showed to be high TNFα producer for both LPS and Candida.

The correlation is calculated by Graphpad software and shown in the figure. From FIG. 5, it can be seen that the cytokine response is different from LPS compared to Candida. This means that the pathways of TLR4 and Dectin-1/MR-1 are different in each individual.

### REFERENCES

1. Siegmund B. Targeted therapies in inflammatory bowel disease. Dig Dis. 2009; 27(4):465-9.
2. Saitoh T, Fujita N, Jang M H, Uematsu S, Yang B G, Satoh T, Omori H, Noda T, Yamamoto N, Komatsu M, Tanaka K, Kawai T, Tsujimura T, Takeuchi O, Yoshimori T, Akira S. Loss of the autophagy protein Atg16L1 enhances endotoxin-induced IL-1beta production. Nature 2009; 456:264-8.
3. Van Limbergen J, Wilson D C, Satsangi J. The genetics of Crohn's disease. Annu Rev Genomics Hum Genet. 2009; 10:89-116
4. Plantinga T S, Crisan T O, Oosting M, van de Veerdonk F L, de Jong D J, Philpott D J, van der Meer J W, Girardin S E, Joosten L A, Netea M G. Crohn's disease-associated ATG16L1 polymorphism modulates pro-inflammatory cytokine responses selectively upon activation of NOD2. Gut. 2011; 60:1229-35.
5. Agostini L, Martinon F, Burns K, McDermott M F, Hawkins P N, Tschopp J. NALP3 forms an IL-1beta-processing inflammasome with increased activity in Muckle-Wells autoinflammatory disorder. Immunity. 2004; 20:319-25.
6. Martinon F, Burns K, Tschopp J. The inflammasome: a molecular platform triggering activation of inflammatory caspases and processing of proIL-beta. Mol Cell. 2002; 10(2):417-26.
7. Dinarello C A. Immunological and inflammatory functions of the interleukin-1 family. Annu Rev Immunol. 2009; 27:519-50.
8. Timmermans K. Blueprint of signaling interactions between Pattern Recognition Receptors: Implications for the design of new Vaccine adjuvants. Clinical and Vaccine Immunology. 2013; 20: 427-432.
9. E Mylona E E, Mouktaroudi M, Crisan T O, Makri S, Pistiki A, Georgitsi M, Savva A, Netea M G, van der Meer J W, Giamarellos-Bourboulis E J, Joosten L A. Enhanced interleukin-1β production of PBMCs from patients with gout after stimulation with Toll-like receptor-2 ligands and urate crystals. Arthritis Res Ther. 2012; 14(4):R158.
10. Joosten L A, Netea M G, Mylona E, Koenders M I, Malireddi R K, Oosting M, Stienstra R, van de Veerdonk F L, Stalenhoef A F, Giamarellos-Bourboulis E J, Kanneganti T D, van der Meer J W. Engagement of fatty acids with Toll-like receptor 2 drives interleukin-1β production via the ASC/caspase 1 pathway in monosodium urate monohydrate crystal-induced gouty arthritis. Arthritis Rheum. 2010 November; 62(11):3237-48.

1. A method for assessing the efficacy of an inhibitor of a pro-inflammatory cytokine in a subject suspected to suffer from an autoimmune and/or inflammatory disease or condition, wherein said pro-inflammatory cytokine is selected from the group consisting of: IL-1-β, IL-6, IL-17, IL-23, IL-12, TNFα, IL-5 and IFNγ and said method comprising the steps of:
(a) obtaining a sample from said subject,
(b1) contacting said sample with a compound able to induce the production of a pro-inflammatory cytokine in said sample and
(b2) contacting said sample with said inhibitor of said pro-inflammatory cytokine in said sample,
(c) determining the expression level of said pro-inflammatory cytokine in said sample at the end of step (b1) and (b2) and
(d) assessing the efficacy of said inhibitor of said pro-inflammatory cytokine as sufficient when at the end of step (b1) a detectable expression level or an increase of the expression level of said pro-inflammatory cytokine has been detected and when at the end of step (b2) a detectable decrease of the expression level of said pro-inflammatory cytokine has been detected.

2. The method according to claim 1, for assessing the efficacy of an inhibitor of B cells in a subject, said method comprising the steps of:
(a) obtaining a sample from said subject,
(b1) contacting said sample with a compound able to induce the production of B cells in said sample and
(b2) contacting said sample with said inhibitor of said B cells in said sample,
(c) determining the number of B cells in said sample at the end of step (b1) and (b2) and
(d) assessing the efficacy of said inhibitor as sufficient when at the end of step (b1) a detectable number or an increase of the number of said B cells has been detected and when at the end of step (b2) a detectable decrease of the number of said B cells has been detected.

3. The method according to claim 1, wherein said method is applied to the following autoimmune and/or inflammatory disease or condition and/or to the following pro-inflammatory cytokine and/or to the following B cell marker:
i. RA and/or the pro-inflammatory cytokine is selected from the group consisting of: TNFα, IL-1β, IL-6, IL-12, IL-17 and IL-23 and/or the B cell marker is CD20 and/or CD19
ii. another RA-like disease and/or the pro-inflammatory cytokine is TNFα,
iii. ulcerative colitis and/or the pro-inflammatory cytokine is TNFα,
iv. Crohn disease and/or the pro-inflammatory cytokine is selected from the group consisting of: TNFα, IL-1β, IL-12, IL-17 and IL-23,
v. psoriasis and/or the pro-inflammatory cytokine is selected from the group consisting of: TNFα, IL-12, IL-17 and IL-23,
vi. MS and/or the pro-inflammatory cytokine is selected from the group consisting of: IL-1β and IL-17 and/or the cell marker is CD20 and/or CD19.
vii. asthma and/or the pro-inflammatory cytokine is selected from the group consisting of: IL-5 and IFNγ,
viii. sepsis and/or the pro-inflammatory cytokine is selected from the group consisting of: IL-1β and IFNγ,
ix. gout and/or the pro-inflammatory cytokine is IL-1β,
x. Lyme disease and/or the pro-inflammatory cytokine is selected from the group consisting of: IL-1β and IL-17,
xi. Type II Diabetes and/or the pro-inflammatory cytokine is selected from the group consisting of: TNFα and IL-1β.

4. The method according to claim 1, wherein the compound is able to induce the production of a pro-inflammatory cytokine in said sample in step (b1) is specific for said autoimmune and/or inflammatory disease or condition.

5. The method according to claim 1, wherein the expression level of said pro-inflammatory cytokine is determined by directly quantifying the amount of said pro-inflammatory cytokine and/or indirectly by quantifying the amount of said nucleotide sequence encoding said pro-inflammatory cytokine.

6. The method according to claim 1, wherein the efficacy of an inhibitor of a pro-inflammatory cytokine is said sufficient when the expression level of said pro-inflammatory cytokine assessed in step (d) following step (b1) has been increased of at least 20% and the expression level of said pro-inflammatory cytokine assessed in step (d) following step (b2) has been decreased of at least 10%.

7. The method according to claim 1, wherein the sample is a fluid obtained from the subject.

8. The method according to claim 7, wherein the fluid is selected from blood or spinal cord fluid.

9. The method according to claim 8, wherein the fluid is blood and comprises PBMC.

10. A method for treating a subject suspected to suffer from an autoimmune and/or inflammatory condition or disease comprising the following steps of assessing the efficacy of an inhibitor of a pro-inflammatory cytokine in a subject as defined in claim 1 and subsequently if the efficacy of said inhibitor is satisfying, treating said subject with said inhibitor.


SEQUENCE LISTING (35 sequences)

SEQ ID NO: 1 - DNA, 14020 bp, Homo sapiens
gtagaatacagcaacgacagacattttgggagagaagcattttatcatagcttttagaagagaagtatttttcagcatcataagcacacaattccaaggacagataccttcaagggattgcttttgacagttatgacaaagtcttaaagaagaataaaaggacaaaggaaatcctccagcaacaaagctgccacttatagatgagaaagtgaatgggaataaggaagaaactcagaaaagggaagagagatcactaaaaaccctgatttggaaagtcccagtactaccctgagaggagaaagaaacaaattcacacagcacgtcaccgccagaagaagaaaggagggaagacaagggaacagagaggatctcaatcctaaaaggacaatgtggaaacatttaggggacagaggtgaatctgccaggccaatgtagtttagaatgtcactcaatcactgagaatgagaaaggagtctgccgatgggcaccatgtggacaggagatgaggcaataaacatatgtaacaattaaaagtgagaaataaagatctggttttggttcagttaacccatatttgagtcgtagttccagcatttactgtttgtgcggtcttaaataatttattgtctcaacctcagtttctgcataggtcaaatggttcaatattatctactttaaaggttattatgaagagttaataagataatgagggaaaaaaaggtacctggcacttagtaggtgctcaataaaggacggctttttttttttaagtattgcttctaaatttgtatgtaagaaaaaatgatataatacaatgatataagacaggggaccgcaggacaagtccagccacacccattcatttatgtattatctgactgcttttctgaaatgatgcaaggttgagtagttgtacctgaaacatttattatctggccctttacagaaaatgtttccagaccctggataagtggtaccagagccccctctgtttgtggtcccctctcttatacccactaggtgtgagaaaagacatagagtaggagagccctgccatccatcttacccacccaggggctttttctgatggatccaaaggaaggacaaggtcttattggtctcccagaactgacataacaactccgacatcagggaaaagccattggagactacatagctcgccagccccagccacctgctcatatatctaagccctccttgttctagaccagggaggagaatggaatgtcccttggactctgcatgtccccaatctgagaacctggatccaagagggagaagaagcccattggagatgatgccataaaggaagtggaagcgatatgataaaaatcatagtgcccattcccaaataatcccagaagcagaagggaaaggagagaaatatccacaaagacaggtgtgggtacacacaacatttttcatactttaagatcccagagggactcatggaaatgatacaagaaaatgactcataagaacaaatattaggaagccagtgccaagaatgagatgggaaattggggaaaatgttgggggcagattgcttagttctgttctaagcaagagggtgaacaaggaaggaacagctcactacaaagaacagacatcactgcatgtacacacaataatataagaactaacccatgattattttgcttgtcttcttgttcaaaatgattgaagaccaatgagatgagatcaaccttgataactggctgcgaagcccatgattagacacaagatggtatcagggcacttgctgctttgaataaatgtcagtctcctgtcttggaagaatgacctgacagggtaaagaggaacttgcagctgagaaaggctttagtgactcaagagctgaataattccccaaaagctggagcatcctggcatttccagctccccatctctgcttgttccacttccttggggctacatcaccatctacatcatcatcactcttccactccctcccttagtgccaactatgtttatagcgagatattttctgctcattggggatcggaaggaagtgctgtggcctgagcggtctccttgggaagacaggatctgatacatacgttgcacaacctatttgacataagaggtttcacttcctgagatggatgggatggtagcagatttgggtccaggttacagggccaggatgagacatggcagaactgtggagactgttacgtcagggggcattgccccatggctccaaaatttccctcgagcgaaagcatcaggggctcatgcaacctggatactagtgctgcttcaaccacactgtgctattggatgagtcacttccaccctcctagccttgatttcttcgtctgctgttcacattcaaatagctattcatgtcttcatctctgtggtcccaccatatcccaccagacaatcattagggctcctcttagctggcagattctgaggtcctggatgctacaattggaagatggagaagtagaagctcaaggtttctgacctgtatcccaagtcccagaagcagaatggactaactcagagctgatgctcgggtcccttgcatatctcccttcctgtcactggctttgatcctccttcgttcagcttgtaatcacatcaacagaccaaagacatctctgtgttctgtcaggagagttcacagagccaccaaccctccagaccctgctggttgccgcataaagactctgaggaagggtttgaggctgctgtgatcatgcaatgaatgcatgattgtaccactgcactccagcctgggggataaaggtagatcctgtctaggagagagagagagagaaagagaaagagagagagaagggagggagagacaaagaaaaagagagagagggagggagaaagaaagagagaaagaaaagagaaaagaaagaaaaagaaagaaagagagagagggagggagggagagagaaagaaagaaagaaagagaaagagagaaagagagaaagagaaagaaaggaagaaagaaagaaagaaagaaagaaagaaagaaagaaagaaagaaaagaaaagaaagaaagagagagagaaagaaaaagaaagaggaaggaaggaaggaaggaagaaagacaggctctgaggaaggtggcagttcctacaacgggagaaccagtggttaatttgcaaagtggatcctgtggaggcaaaacagaggagtcccctaggccacccagacagggcttttagctatctgcaggaccagacaccaaatttcaggagggctcagtgttaggaatggattatggcttatcaaattcacaggaaactaacatgttgaacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttggataatgcagagcgagcacgatacctggcacatactaatttgaataaaaatgctgtcaaattcccattcacccattcaagcagcaaactctaccacctgaatgtacatgccaggcactgtgctagacttggctcaaaaagatttcagtttcctggaggaaccaggaggagcaaggtttcaactcagtgctataagaagtgttacaggctggacacggtggctcacgcctgtaatcccaacactttgggaggccgaggcgggcagatcacaaggtcaggagatcgagaccatcctggctaacatggtgaaaccctgtctctactaaaaatacaaaaaattagccgggcgtggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaagtgttatgatgcagacctgtcaaagaggcaaaggagggtgttcctacactccaggcactgttcataacctggactctcattcattctacaaatggagggctcccctgggcagtaccctggagcaggcactttgctggtgtctcggttaaagagaaactgataactcttggttggtattaccaagagatagagtctcagatggatattcttacagaaacaatattccacttttcagagttcaccaaaaaatcattttaggcagagctcatctggcattgatctggttcatccatgagattggctagggtaacagcacctggtcttgcagggttgtgtgagcttatctccagggttgccccaactccgtcaggagcctgaaccctgcataccgtatgttctctgccccagccaagaaaggtcaattttctcctcagaggctcctgcaattgacagagagctcctgaggcagagaacagcacccaaggtagagacccacaccctcaatacagacagggagggctattggcccttcattgtacccatttatccatctgtaagtgggaagattcctaaacttaagtacaaagaagtgaatgaagaaaagtatgtgcatgtataaatctgtgtgtcttccactttgtcccacatatactaaatttaaacattcttctaacgtgggaaaatccagtattttaatgtggacatcaactgcacaacgattgtcaggaaaacaatgcatatttgcatggtgatacatttgcaaaatgtgtcatagtttgctactccttgcccttccatgaaccagagaattatctcagtttattagtcccctcccctaagaagcttccaccaatactcttttcccctttcctttaacttgattgtgaaatcaggtattcaacagagaaatttctcagcctcctacttctgcttttgaaagccataaaaacagcgagggagaaactggcagataccaaacctcttcgaggcacaaggcacaacaggctgctctgggattctcttcagccaatcttcattgctcaagtatgactttaatcttccttacaactaggtgctaagggagtctctctgtctctctgcctctttgtgtgtatgcatattctctctctctctctctttctttctctgtctctccctctccttccctctctgcctccctctctcagctttttgcaaaaatgccaggtgtaatataatgcttatgactcgggaaatattctgggaatggatactgcttatctaacagctgacaccctaaaggttagtgtcaaagcctctgctccagctctcctagccaatacattgctagttggggtttggtttagcaaatgcttttctctagacccaaaggacttctctttcacacattcattcatttactcagagatcatttctttgcatgactgccatgcactggatgctgagagaaatcacacatgaacgtagccgtcatggggaagtcactcattttctcctttttacacaggtgtctgaagcagccatggcagaagtacctgagctcgccagtgaaatgatggcttattacaggtcagtggagacgctgagaccagtaacatgagcaggtctcctctttcaagagtagagtgttatctgtgcttggagaccagatttttcccctaaattgcctctttcagtggcaaacagggtgccaagtaaatctgatttaaagactactttcccattacaagtccctccagccttgggacctggaggctatccagatgtgttgttgcaagggcttcctgcagaggcaaatggggagaaaagactccaagcccacaatacaaggaatccctttgcaaagtgtggcttggagggagagggagagctcagattttagctgactctgctgggctagaggttaggcctcaagatccaacagggagcacccagggtgcccacctgccaggcctagaatctgccttctggactgttctgcgcatatcactgtgaaacttgccaggtgtttcaggcagctttgagaggcaggctgtttgcagtttcttatgaacagtcaagtcttgtacacagggaaggaaaaataaacctgtttagaagacataattgagacatgtccctgtttttattacagtggcaatgaggatgacttgttctttgaagctgatggccctaaacagatgaaggtaagactatgggtttaactcccaacccaaggaagggctctaacacagggaaagctcaaagaagggagttctgggccactttgatgccatggtattttgttttagaaagactttaacctcttccagtgagacacaggctgcaccacttgctgacctggccacttggtcatcatatcaccacagtcactcactaacgttggtggtggtggccacacttggtggtgacaggggaggagtagtgataatgtttcccatttcatagtaggaagacaaccaagtcttcaacataaatttgattatccttttaagagatggattcagcctatgccaatcacttgagttaaactctgaaaccaagagatgatcttgagaactaacatatgtctaccccttttgagtagaatagttttttgctacctggggtgaagcttataacaacaagacatagatgatataaacaaaaagatgaattgagacttgaaagaaaaccattcacttgctgtttgaccttgacaagtcattttacccgctttggacctcatctgaaaaataaagggctgagctggatgatctctgagattccagcatcctgcaacctccagttctgaaatattttcagttgtagctaagggcatttgggcagcaaatggtcatttttcagactcatccttacaaagagccatgttatattcctgctgtcccttctgttttatatgatgctcagtagccttcctaggtggcccagccatcagcctagctaggtcagttgtgcaggttgggaggcagccacttttctctggctttattttattccagtttgtgatagcctcccctagcctcataatccagtcctcaatcttgttaaaaacatatttctttagaagttttaagactggcataacttgttggctgcagctgtgggaggagcccattggcttgtctgcctggcctttgcccccattgcctcttccagcagcttggctctgctccaggcaggaaattctctcctgctcaactttcttttgtgcacttacaggtctctttaactgtctttcaagcctttgaaccattatcatgccttaaggcaacctcagtgaagccttaatacggagcttctctgaataagaggaaagtggtaacatttcacaaaaagtactctcacaggatttgcagaatgcctatgagacagtgttatgaaaaaggaaaaaaaagaacagtgtagaaaaattgaatacttgctgagtgagcataggtgaatggaaaatgttatggtcatctgcatgaaaaagcaaatcatagtgtgacagcattagggatacaaaaagatatagagaaggtatacatgtatggtgtaggtggggcatgtacaaaaaagatgaacaaagtagaaatgggatttattctaaaagaatagcctgtaaggtgtcagaaagcccacattctagtcttgagtctgcctctaacctgctgtgtgcccttgagtacacacttaacctccttgagcttcagagagggataatctttttattttattttattttattttgttttgttttgttttgttttgttttatgagacagagtctcactctgttgcccaggctggagtgcagtggtacaatcttggcttactgcatcctccacctcctgagttcaagcgattctccttcctcagtctcctgaatagctaggattacaggtgcaccccaccacacccagctaatttttgtatttttagtagagaaggggtttcgccatgttggccaggctggttttgaagtcctgacctaaatgattcatccacctcggcttcccaaagtgctgggattacaggcatgagccaccacgcctggcccagagagggatgatctttagaagctcgggattctttcaagccctttcctcctctctgagctttctactctctgatgtcaaagcatggttcctggcaggaccacctcaccaggctccctccctcgctctctccgcagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaaggtagttagccaagagcaggcagtagatctccacttgtgtcctcttggaagtcatcaagccccagccaactcaattcccccagagccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccgcctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccacccaagtctcttgggcatagtgcaatgtcaattcttccacaatatggggtcatttgatggacatggcctaactgcctgtgggttctctcttcctgttgttgaggctgaaacaagagtgctggagcgataatgtgtccatccccctccccagtcttccccccttgccccaacatccgtcccacccaatgccaggtggttccttgtagggaaattttaccgcccagcaggaacttatatctctccgctgtaacgggcaaaagtttcaagtgcggtgaacccatcattagctgtggtgatctgcctggcatcgtgccacagtagccaaagcctctgcacaggagtgtgggcaactaaggctgctgactttgaaggacagcctcactcagggggaagctatttgctctcagccaggccaagaaaatcctgtttctttggaatcgggtagtaagagtgatcccagggcctccaattgacactgctgtgactgaggaagatcaaaatgagtgtctctctttggagccactttcccagctcagcctctcctctcccagtttcttcccatgggctactctctgttcctgaaacagttctggtgcctgatttctggcagaagtacagcttcacctctttcctttccttccacattgatcaagttgttccgctcctgtggatgggcacattgccagccagtgacacaatggcttccttccttccttccttcagcatttaaaatgtagaccctctttcattctccgttcctactgctatgaggctctgagaaaccctcaggcctttgaggggaaaccctaaatcaacaaaatgaccctgctattgtctgtgagaagtcaagttatcctgtgtcttaggccaaggaacctcactgtgggttcccacagaggctaccaaattacatgtatcctactcatggggcctaggggttggggtgaccctgcactgctgtgtccctaaccacaagacccccttctttcttcagtggtgttctccatgtcctttgtacaaggagaagaaagtaatgacaaaatacctgtggccttgggcctcaaggaaaagaatctgtacctgtcctgcgtgttgaaagatgataagcccactctacagctggaggtaagtgaatgctatggaatgaagcccttctcagcctcctgctaccacttattcccagacaaccaccttctccccgcccccatccctaggaaaagctgggaacaggtctatttgacaattttgcattaatgtaaataaatttaacataatttttaactgcgtgcaaccttcaatcctgctgcagaaaattaaatcattttgccgatgttattatgtcctaccatagttacaaccccaacagattatatattgttagggctgctctcatttgatagacaccttgggaaatagatgacttaaagggtcccattatcatgtccactccactcccaaaattaccaccactatcacctccagctttctcagcaaaagcttcatttccaagttgatgtcattctaggaccataaggaaaaatacaataaaaagcccctggaaactaggtacttcaagaagctctagcttaattttcacccccccaaaaaaaaaaaattctcacctacattatgctcctcagcatttggcactaagttttagaaaagaagaagggctcttttaataatcacacggaaagttgggggcccagttacaactcaggagtctggctcctgatcatgtgacctgctcgtcagtttcctttctggccaacccaaagaacatctttcccatagcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaagaaaaaaaaagggtctttcctgatcattgacttgtcttggatttgacactgaacagtaaagacaaacagggctgtgagagttcttgggggactaaagcccactcctcattgctgagtgctgcaaagtacctagaaatatccttggccaccgaagactatcctcctcacccatcccctttatttctgttgttcaacagaaggatattcagtgcacatttggaacaggatcagctgaagcactgcagggagtcaggactggtagtaacagctaccagtgatttatctatcaatgcaccaaacatctgttgagcaagcgctatgtactaggagctgggagtacagagatgagaacagtcacaagtccctcctcagataggagaggcagctagttataagcagaaacaaggtaacatgacaagtagagtaagataaagaacaagaggagtagccaggaaggagggaggagaacgacataagaatcaagcctaaagggataaacagaagatttccacacatgggctgggcatggtggcttacgcctgtaatcccagcactttgggtggcaggggcagaaagatcgcttgagcccaggagttcaagaccagcctgggcaacatagtgagactcccatctctacaaaaaataaataaataaataaaacaatcagccaggcatgctggcatgcacctgtagtcctagctacttgggaagctgacactggaggattgcttgagcccagaagttcaagactgcagtgagctgtgatcgcaccattgcactccagcctgggtgacagagtgagaccctgtctctaaaaaatgttcccagatagaaaagaaaagaaaaagccctcaggtagaggaaccagtgtgaacaagagcatgggtgtataagaatgaatgatgcaaaaagtacactttcagaattgcgggaatcaccagaagcaaagtcaaagcgcaaaacaagccagaggaaaagtagttgtaactcacaccccagataaagagataacttgtttcacatgtaaagaactgcctcaaaacatttgaaaaaaaagaacaaaaatccagcaaaacaagaggcaagggatcttaacagtctgttcacagaaaagaaaatactatttgatcttgggcagagtaaaatgatgcttaacattgtaataagaaagtcaaattaaaagcactttgagatactagtattttcccatcagattgacaaaaatcaaaagtttaacaacagaccttgttggtcaaactgtcgggaaatccccactgttaaatattacgcgtgggactataaattgatatgacccttatagaacaaaatttgctaactatgaaaatcacaagtgcacttcccctttgatccagcaatttcactcctggagatttatcccacagatggacataacccatgtgaaatggaaaatgatcaaaattattcattgcacatcatttgtaataggaaaaattggaagtaacccaagtgtctatcaacaagagactgcctaaatgaagtaaaggacatagaatactaggcagctatagaaaagaatgagaaagcactctggtattgtttggttctgtgtcccagcccaaatctcatgtcaaattgtaatccccgatgttggaggtggggcctggtgtggggtgattggatcatgggggtggagttcttatgaatggtttagcactatccctttggtgctgttctcgtgacagagttcccacaagatctggttgtttaaaagtatgtggcatcctttctctctctctctctctctctcagtcctgctcctgccatataagacatccactcccgctttgtcttctgcatgagtaaaagcttcctaaggcctccccagaagcagatgctgccatgcttcctgtggaacagcctgcgaagctgtgagccaattaaacctcttttctttataaattatgcagtctcaggtatttccttatagcaatgcaaggactgactaatacatgctgtctg

SEQ ID NO: 2 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 3 - DNA, 639 bp, Homo sapiens
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtag

SEQ ID NO: 4 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 5 - DNA, 468 bp, Homo sapiens
atgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaa

SEQ ID NO: 6 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 7 - DNA, 570 bp, Homo sapiens
atgctggggagcagagctgtaatgctgctgttgctgctgccctggacagctcagggcagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtccctaa

SEQ ID NO: 8 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 9 - DNA, 702 bp, Homo sapiens
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 10 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 11 - DNA, 501 bp, Homo sapiens
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 12 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 13 - DNA, 21 bp, Artificial
  forward primer IL-1beta
cagctacgaatctccgaccac

SEQ ID NO: 14 - DNA, 20 bp, Artificial
  reverse primer IL-1beta
ggcagggaaccagcatcttc

SEQ ID NO: 15 - DNA, 21 bp, Artificial
  forward primer IL-6
aattcggtacatcctcgacgg

SEQ ID NO: 16 - DNA, 21 bp, Artificial
  Reverse primer IL-6
ggttgttttctgccagtgcct

SEQ ID NO: 17 - DNA, 20 bp, Artificial
  Forward primer IL-17
tcccacgaaatccaggatgc

SEQ ID NO: 18 - DNA, 22 bp, Artificial
  reverse primer IL-17
ggatgttcaggttgaccatcac

SEQ ID NO: 19 - DNA, 21 bp, Artificial
  Forward primer IL-23
ctcagggacaacagtcagttc

SEQ ID NO: 20 - DNA, 19 bp, Artificial
  Reverse primer IL-23
acagggctatcagggagca

SEQ ID NO: 21 - DNA, 17 bp, Artificial
  Forward primer TNFalpha
tggcccaggcagtcaga

SEQ ID NO: 22 - DNA, 23 bp, Artificial
  Reverse primer TNFalpha
ggtttgctacaacatgggctaca

SEQ ID NO: 23 - DNA, 23 bp, Artificial
  Forward primer IFNgamma
tcggtaactgacttgaatgtcca

SEQ ID NO: 24 - DNA, 22 bp, Artificial
  Reverse primer IFNgamma
tcctttttcgcttccctgtttt

SEQ ID NO: 25 - DNA, 762 bp, Homo sapiens
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 26 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 27 - DNA, 23 bp, Artificial
  Forward primer IL-12
ccttgcacttctgaagagattga

SEQ ID NO: 28 - DNA, 22 bp, Artificial
  Reverse primer IL-12
acagggccatcataaaagaggt

SEQ ID NO: 29 - DNA, 816 bp, Homo sapiens
atgcactttctttgccaaaggcaaacgcagaacgtttcagagccatgaggatgcttctgcatttgagtttgctagctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttgagactaaactggtttgttgcagccaaagattttggaggagaaggacattttactgcagtgagaatgagggccaagaaagagtcaggccttaattttcagtataatttaacttcagagggaaagtaaatatttcaggcatactgacactttgccagaaagcataaaattcttaaaatatatttcagatatcagaatcattgaagtattttcctccaggcaaaattgatatacttttttcttatttaacttaacattctgtaaaatgtctgttaacttaatagtatttatgaaatggttaagaatttggtaaattagtatttatttaatgttatgttgtgttctaataaaacaaaaatagacaactgttc

SEQ ID NO: 30 - protein, 133 aa, Homo sapiens
MRRMLLHLSVLTLSCVWATAMEIPMSTVVKETLTQLSAHRALLTSNETMRLPVPTHKNHQLCIGEIFQGLDILKNQTVRGGTVEMLFQNLSLIKKYIDRQKEKCGEERRRTRQFLDYLQEFLGVMSTEWAMEG

SEQ ID NO: 31 - DNA, 22 bp, Artificial
  Forward primer IL-5
tctactcatcgaactctgctga

SEQ ID NO: 32 - DNA, 21 bp, Artificial
  Reverse primer IL-5
cccttgcacagtttgactctc

SEQ ID NO: 33 - protein, 21 aa, Artificial
  MOG peptides
MEVGWYRSPFSRVVHLYRNGK

SEQ ID NO: 34 - DNA, 23 bp, Artificial
  Primer
ccttgcacttctgaagagattga

SEQ ID NO: 35 - DNA, 22 bp, Artificial
  Primer
acagggccatcataaaagaggt